SEQUENCE LISTING (16 sequences)

SEQ ID NO: 1 - DNA, 2478 bp, Bifidobacterium adolescentis
  CDS (1)..(2478)
atgacgagtcctgttattggcaccccttggaagaagctgaacgctccggtttccgaggaagctatcgaaggcgtggataagtactggcgcgcagccaactacctctccatcggccagatctatctgcgtagcaacccgctgatgaaggagcctttcacccgcgaagacgtcaagcaccgtctggtcggtcactggggcaccaccccgggcctgaacttcctcatcggccacatcaaccgtctcattgctgatcaccagcagaacactgtgatcatcatgggcccgggccacggcggcccggctggtaccgctcagtcctacctggacggcacctacaccgagtacttcccgaacatcaccaaggatgaggctggcctgcagaagttcttccgccagttctcctacccgggtggcatcccgtcccactacgctccggagaccccgggctccatccacgaaggcggcgagctgggttacgccctgtcccacgcctacggcgctgtgatgaacaacccgagcctgttcgtcccggccatcgtcggcgacggtgaagctgagaccggcccgctggccaccggctggcagtccaacaagctcatcaacccgcgcaccgacggtatcgtgctgccgatcctgcacctcaacggctacaagatcgccaacccgaccatcctgtcccgcatctccgacgaagagctccacgagttcttccacggcatgggctatgagccgtacgagttcgtcgctggcttcgacaacgaggatcacctgtcgatccaccgtcgtttcgccgagctgttcgagaccgtcttcgacgagatctgcgacatcaaggccgccgctcagaccgacgacatgactcgtccgttctacccgatgatcatcttccgtaccccgaagggctggacctgcccgaagttcatcgacggcaagaagaccgagggctcctggcgttcccaccaggtgccgctggcttccgcccgcgataccgaggcccacttcgaggtcctcaagaactggctcgagtcctacaagccggaagagctgttcgacgagaacggcgccgtgaagccggaagtcaccgccttcatgccgaccggcgaactgcgcatcggtgagaacccgaacgccaacggtggccgcatccgcgaagagctgaagctgccgaagctggaagactacgaggtcaaggaagtcgccgagtacggccacggctggggccagctcgaggccacccgtcgtctgggcgtctacacccgcgacatcatcaagaacaacccggactccttccgtatcttcggaccggatgagaccgcttccaaccgtctgcaggccgcttacgacgtcaccaacaagcagtgggacgccggctacctgtccgctcaggtcgacgagcacatggctgtcaccggccaggtcaccgagcagctttccgagcaccagatggaaggcttcctcgagggctacctgctgaccggccgtcacggcatctggagctcctatgagtccttcgtgcacgtgatcgactccatgctgaaccagcacgccaagtggctcgaggctaccgtccgcgagattccgtggcgcaagccgatctcctccatgaacctgctcgtctcctcccacgtgtggcgtcaggatcacaacggcttctcccaccaggatccgggtgtcacctccgtcctgctgaacaagtgcttcaacaacgatcacgtgatcggcatctacttcccggtggattccaacatgctgctcgctgtggctgagaagtgctacaagtccaccaacaagatcaacgccatcatcgccggcaagcagccggccgccacctggctgaccctggacgaagctcgcgccgagctcgagaagggtgctgccgagtggaagtgggcttccaacgtgaagtccaacgatgaggctcagatcgtgctcgccgccaccggtgatgttccgactcaggaaatcatggccgctgccgacaagctggacgccatgggcatcaagttcaaggtcgtcaacgtggttgacctggtcaagctgcagtccgccaaggagaacaacgaggccctctccgatgaggagttcgctgagctgttcaccgaggacaagccggtcctgttcgcttaccactcctatgcccgcgatgtgcgtggtctgatctacgatcgcccgaaccacgacaacttcaacgttcacggctacgaggagcagggctccaccaccaccccgtacgacatggttcgcgtgaacaacatcgatcgctacgagctccaggctgaagctctgcgcatgattgacgctgacaagtacgccgacaagatcaacgagctcgaggccttccgtcaggaagccttccagttcgctgtcgacaacggctacgatcacccggattacaccgactgggtctactccggtgtcaacaccaacaagcagggtgctatctccgctaccgccgcaaccgctggcgataacgagtga

SEQ ID NO: 2 - protein, 825 aa, Bifidobacterium adolescentis
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDNGYDHPDYTDWVYSGVNTNKQGAISATAATAGDNE

SEQ ID NO: 3 - DNA, 999 bp, Escherichia coli
  CDS (1)..(999)
atgaaaacgttaggtgaatttattgtcgaaaagcagcacgagttttctcatgctaccggtgagctcactgctttgctgtcggcaataaaactgggcgccaagattatccatcgcgatatcaacaaagcaggactggttgatatcctgggtgccagcggtgctgagaacgtgcagggcgaggttcagcagaaactcgacttgttcgctaatgaaaaactgaaagccgcactgaaagcacgcgatatcgttgcgggcattgcctctgaagaagaagatgagattgtcgtctttgaaggctgtgaacacgcaaaatacgtggtgctgatggaccccctggatggctcgtccaacatcgatgttaacgtctctgtcggtaccattttctccatctaccgccgcgttacgcctgttggcacgccggtaacggaagaagatttcctccagcctggtaacaaacaggttgcggcaggttacgtggtatacggctcctctaccatgctggtttacaccaccggatgcggtgttcacgcctttacttacgatccttcgctcggcgttttctgcctgtgccaggaacggatgcgcttcccggagaaaggcaaaacctactccatcaacgaaggaaactacattaagtttccgaacggggtgaagaagtacattaaattctgccaggaagaagataaatccaccaaccgcccttatacctcacgttatatcggttcactggtcgcggatttccaccgtaacctgctgaaaggcggtatttatctctacccaagcaccgccagccacccggacggcaaactgcgtttgctgtatgagtgcaacccgatggcattcctggcggaacaagcgggcggtaaagcgagcgatggcaaagagcgtattctggatatcatcccggaaaccctgcaccagcgccgttcattctttgtcggcaacgaccatatggttgaagatgtcgaacgctttatccgtgagttcccggacgcgtaa

SEQ ID NO: 4 - protein, 332 aa, Escherichia coli
MKTLGEFIVEKQHEFSHATGELTALLSAIKLGAKIIHRDINKAGLVDILGASGAENVQGEVQQKLDLFANEKLKAALKARDIVAGIASEEEDEIVVFEGCEHAKYVVLMDPLDGSSNIDVNVSVGTIFSIYRRVTPVGTPVTEEDFLQPGNKQVAAGYVVYGSSTMLVYTTGCGVHAFTYDPSLGVFCLCQERMRFPEKGKTYSINEGNYIKFPNGVKKYIKFCQEEDKSTNRPYTSRYIGSLVADFHRNLLKGGIYLYPSTASHPDGKLRLLYECNPMAFLAEQAGGKASDGKERILDIIPETLHQRRSFFVGNDHMVEDVERFIREFPDA

SEQ ID NO: 5 - DNA, 678 bp, Escherichia coli
  CDS (1)..(678)
atgaaacagtatttgattgccccctcaattctgtcggctgattttgcccgcctgggtgaagataccgcaaaagccctggcagctggcgctgatgtcgtgcattttgacgtcatggataaccactatgttcccaatctgacgattgggccaatggtgctgaaatccttgcgtaactatggcattaccgcccctatcgacgtacacctgatggtgaaacccgtcgatcgcattgtgcctgatttcgctgccgctggtgccagcatcattacctttcatccagaagcctccgagcatgttgaccgcacgctgcaactgattaaagaaaatggctgtaaagcgggtctggtatttaacccggcgacacctctgagctatctggattacgtgatggataagctggatgtgatcctgctgatgtccgtcaaccctggtttcggcggtcagtctttcattcctcaaacactggataaactgcgcgaagtacgtcgccgtatcgacgagtctggctttgacattcgactagaagtggacggtggcgtgaaggtgaacaacattggcgaaatcgctgcggcgggcgcggatatgttcgtcgccggttcggcaatcttcgaccagccagactacaaaaaagtcattgatgaaatgcgcagtgaactggcaaaggtaagtcatgaataa

SEQ ID NO: 6 - protein, 225 aa, Escherichia coli
MKQYLIAPSILSADFARLGEDTAKALAAGADVVHFDVMDNHYVPNLTIGPMVLKSLRNYGITAPIDVHLMVKPVDRIVPDFAAAGASIITFHPEASEHVDRTLQLIKENGCKAGLVFNPATPLSYLDYVMDKLDVILLMSVNPGFGGQSFIPQTLDKLREVRRRIDESGFDIRLEVDGGVKVNNIGEIAAAGADMFVAGSAIFDQPDYKKVIDEMRSELAKVSHE

SEQ ID NO: 7 - DNA, 660 bp, Escherichia coli
  CDS (1)..(660)
atgacgcaggatgaattgaaaaaagcagtaggatgggcggcacttcagtatgttcagcccggcaccattgttggtgtaggtacaggttccaccgccgcacactttattgacgcgctcggtacaatgaaaggccagattgaaggggccgtttccagttcagatgcttccactgaaaaactgaaaagcctcggcattcacgtttttgatctcaacgaagtcgacagccttggcatctacgttgatggcgcagatgaaatcaacggccacatgcaaatgatcaaaggcggcggcgcggcgctgacccgtgaaaaaatcattgcttcggttgcagaaaaatttatctgtattgcagacgcttccaagcaggttgatattctgggtaaattcccgctgccagtagaagttatcccgatggcacgtagtgcagtggcgcgtcagctggtgaaactgggcggtcgtccggaataccgtcagggcgtggtgaccgataatggcaacgtgatcctcgacgtccacggcatggaaatccttgacccgatagcgatggaaaacgccataaatgcgattcctggcgtggtgactgttggcttgtttgctaaccgtggcgcggacgttgcgctgattggcacacctgacggtgtcaaaaccattgtgaaatga

SEQ ID NO: 8 - protein, 219 aa, Escherichia coli
MTQDELKKAVGWAALQYVQPGTIVGVGTGSTAAHFIDALGTMKGQIEGAVSSSDASTEKLKSLGIHVFDLNEVDSLGIYVDGADEINGHMQMIKGGGAALTREKIIASVAEKFICIADASKQVDILGKFPLPVEVIPMARSAVARQLVKLGGRPEYRQGVVTDNGNVILDVHGMEILDPIAMENAINAIPGVVTVGLFANRGADVALIGTPDGVKTIVK

SEQ ID NO: 9 - DNA, 954 bp, Escherichia coli
  CDS (1)..(954)
atgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgtctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccaggatccaatggcagtagataaactggcggaaggtatccgtaagtttgctattgaccaggaaaaactggaaaaaatgatcggcgatctgctgtaa

SEQ ID NO: 10 - protein, 317 aa, Escherichia coli
MTDKLTSLRQYTTVVADTGDIAAMKLYQPQDATTNPSLILNAAQIPEYRKLIDDAVAWAKQQSNDRAQQIVDATDKLAVNIGLEILKLVPGRISTEVDARLSYDTEASIAKAKRLIKLYNDAGISNDRILIKLASTWQGIRAAEQLEKEGINCNLTLLFSFAQARACAEAGVFLISPFVGRILDWYKANTDKKEYAPAEDPGVVSVSEIYQYYKEHGYETVVMGASFRNIGEILELAGCDRLTIAPALLKELAESEGAIERKLSYTGEVKARPARITESEFLWQHNQDPMAVDKLAEGIRKFAIDQEKLEKMIGDLL

SEQ ID NO: 11 - DNA, 1992 bp, Escherichia coli
  CDS (1)..(1992)
atgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 12 - protein, 663 aa, Escherichia coli
MSSRKELANAIRALSMDAVQKAKSGHPGAPMGMADIAEVLWRDFLKHNPQNPSWADRDRFVLSNGHGSMLIYSLLHLTGYDLPMEELKNFRQLHSKTPGHPEVGYTAGVETTTGPLGQGIANAVGMAIAEKTLAAQFNRPGHDIVDHYTYAFMGDGCMMEGISHEVCSLAGTLKLGKLIAFYDDNGISIDGHVEGWFTDDTAMRFEAYGWHVIRDIDGHDAASIKRAVEEARAVTDKPSLLMCKTIIGFGSPNKAGTHDSHGAPLGDAEIALTREQLGWKYAPFEIPSEIYAQWDAKEAGQAKESAWNEKFAAYAKAYPQEAAEFTRRMKGEMPSDFDAKAKEFIAKLQANPAKIASRKASQNAIEAFGPLLPEFLGGSADLAPSNLTLWSGSKAINEDAAGNYIHYGVREFGMTAIANGISLHGGFLPYTSTFLMFVEYARNAVRMAALMKQRQVMVYTHDSIGLGEDGPTHQPVEQVASLRVTPNMSTWRPCDQVESAVAWKYGVERQDGPTALILSRQNLAQQERTEEQLANIARGGYVLKDCAGQPELIFIATGSEVELAVAAYEKLTAEGVKARVVSMPSTDAFDKQDAAYRESVLPKAVTARVAVEAGIADYWYKYVGLNGAIVGMTTFGESAPAELLFEEFGFTVDNVVAKAKELL

SEQ ID NO: 13 - DNA, 768 bp, Escherichia coli
  CDS (1)..(768)
atgcgacatcctttagtgatgggtaactggaaactgaacggcagccgccacatggttcacgagctggtttctaacctgcgtaaagagctggcaggtgttgctggctgtgcggttgcaatcgcaccaccggaaatgtatatcgatatggcgaagcgcgaagctgaaggcagccacatcatgctgggtgcgcaaaacgtggacctgaacctgtccggcgcattcaccggtgaaacctctgctgctatgctgaaagacatcggcgcacagtacatcatcatcggtcactctgaacgtcgtacttaccacaaagaatctgacgaactgatcgcgaaaaaattcgcggtgctgaaagagcagggcctgactccggttctgtgcatcggtgaaaccgaagctgaaaatgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccggatatcgacggcgcgctggttggtggtgcttctctgaaagctgacgccttcgcagtaatcgttaaagctgcagaagcggctaaacaggcttaa

SEQ ID NO: 14 - protein, 255 aa, Escherichia coli
MRHPLVMGNWKLNGSRHMVHELVSNLRKELAGVAGCAVAIAPPEMYIDMAKREAEGSHIMLGAQNVDLNLSGAFTGETSAAMLKDIGAQYIIIGHSERRTYHKESDELIAKKFAVLKEQGLTPVLCIGETEAENEAGKTEEVCARQIDAVLKTQGAAAFEGAVIAYEPVWAIGTGKSATPAQAQAVHKFIRDHIAKVDANIAEQVIIQYGGSVNASNAAELFAQPDIDGALVGGASLKADAFAVIVKAAEAAKQA

SEQ ID NO: 15 - DNA, 1080 bp, Escherichia coli
  CDS (1)..(1080)
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaa

SEQ ID NO: 16 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

(19) United States
(12) Patent Application Publication
(10) Pub. No.: US 2014/0273164 A1
(43) Pub. Date: Sep. 18, 2014

(54) NON-CO2 EVOLVING METABOLIC PATHWAY FOR CHEMICAL PRODUCTION

(71) Applicant: The Regents of the University of California, Oakland, CA (US)

(72) Inventors: James C. Liao, Los Angeles, CA (US); Igor Bogorad, Tarzana, CA (US)

(73) Assignee: The Regents of the University of California, Oakland, CA (US)

(21) Appl. No.: 14/211,337

(22) Filed: Mar. 14, 2014

Related U.S. Application Data
(60) Provisional application No. 61/785,254, filed on Mar. 14, 2013.

(51) Int. Cl. C12N 15/70

(52) U.S. Cl. CPC C12N 15/70
USPC 435/252.33; 435/254.2

(57) ABSTRACT

Provided are microorganisms that catalyze the synthesis of chemicals and biochemicals from a suitable carbon source. Also provided are methods of generating such organisms and methods of synthesizing chemicals and biochemicals using such organisms.

### CROSS REFERENCE TO RELATED APPLICATIONS

This application claims priority to U.S. Provisional Application Ser. No. 61/785,254, filed Mar. 14, 2013, the disclosure of which is incorporated herein by reference in its entirety.

### TECHNICAL FIELD

Metabolically-modified microorganisms and methods of producing such organisms are provided. Also provided are methods of producing chemicals by contacting a suitable substrate with a metabolically-modified microorganism and enzymatic preparations of the disclosure.

### BACKGROUND

Acetyl-CoA is a central metabolite key to both cell growth as well as biosynthesis of multiple cell constituents and products, including fatty acids, amino acids, isoprenoids, and alcohols. Typically, the Embden-Meyerhof-Parnas (EMP) pathway, the Entner-Doudoroff (ED) pathway, and their variations are used to produce acetyl-CoA from sugars through oxidative decarboxylation of pyruvate. Similarly, the CBB, RuMP, and DHA pathways incorporate C1 compounds, such as CO₂ and methanol, to synthesize sugar-phosphates and pyruvate, which then produce acetyl-CoA through decarboxylation of pyruvate. Thus, in all heterotrophic organisms and those autotrophic organisms that use the sugar-phosphate-dependent pathways for C1 incorporation, acetyl-coA is derived from oxidative decarboxylation of pyruvate, resulting in loss of one molecule of CO₂ per molecule of pyruvate. While the EMP route to acetate and ethanol has been optimized, the CO₂ loss problem has not been solved due to inherent pathway limitations. Without using a CO₂ fixation pathway, such as the Wood-Ljungdahl pathway or the reductive TCA cycle, the waste CO₂ leads to a significant decrease in carbon yield. This loss of carbon has a major impact on the overall economy of biorefinery and the carbon efficiency of cell growth.

### SUMMARY

For industrial applications, the carbon utilization pathway of the disclosure can be used to improve carbon yield in the production of fuels and chemicals derived from acetyl-CoA, such as, but not limited to, acetate, n-butanol, isobutanol, ethanol and the like. For example, if additional reducing power such as hydrogen or formic acid is provided, the carbon utilization pathway of the disclosure can be used to produce compounds that are more reduced than the substrate, for example, ethanol, 1-butanol, isoprenoids, and fatty acids from sugar. When the pathway is combined with the RuMP pathway, it can convert methanol to ethanol or butanol.

The disclosure provides a recombinant microorganism comprising a non-CO₂ evolving metabolic pathway for the synthesis of acetyl phosphate with improved carbon yield beyond 1:2 molar ratio (fructose 6-phosphate:Acetyl phosphate) from a carbon substrate using a pathway comprising an enzyme having fructose-6-phosphoketolase (Fpk) activity and/or xylulose-5-phosphoketolase (Xpk) activity. In one embodiment, the microorganism can convert any sugar phosphate to acetyl phosphate with improved yield beyond those obtained by pathways that involve pyruvate decarboxylation. In another embodiment, the sugar phosphate is selected from the group consisting of: sugar phosphates of a triose (G3P, DHAP), an erythrose (E4P), a pentose (R5P, Ru5P, RuBP, X5P), a hexose (F6P, H6P, FBP, G6P), and a sedoheptulose (S7P, SBP). In another embodiment, the sugar phosphates are derived from methanol, methane, CO₂, CO, formaldehyde, formate, glycerol, a carbohydrate having the general formula CₙH₂ₙOₙ, wherein n=3 to 7, or cellulose as a carbon source. In another embodiment, the microorganism is a prokaryote or eukaryote. In another embodiment, the microorganism is yeast. In another embodiment, the microorganism is a prokaryote. In another embodiment, the microorganism is derived from an E. coli microorganism. In another embodiment, an E. coli is engineered to express a phosphoketolase. In another embodiment, the phosphoketolase is Fpk, Xpk or a bifunctional F/Xpk enzyme. In any of the foregoing embodiments, the microorganism is engineered to heterologously express one or more of the following enzymes: (a) a phosphoketolase (F/Xpk); (b) a transaldolase (Tal); (c) a transketolase (Tkt); (d) a ribose-5-phosphate isomerase (Rpi); (e) a ribulose-5-phosphate epimerase (Rpe); (f) a triose phosphate isomerase (Tpi); (g) a fructose 1,6 bisphosphate aldolase (Fba); (h) a sedoheptulose bisphosphate aldolase (Sba); (i) a fructose 1,6 bisphosphatase (Fbp); and (j) a sedoheptulose 1,6, bisphosphatase (Sbp). In any of the foregoing embodiments, the microorganism is engineered to express a phosphoketolase derived from Bifidobaceterium adolescentis. In another embodiment, the phosphoketolase comprises a sequence that is at least 49% identical to SEQ ID NO:2 and has phosphoketolase activity. In another embodiment, the microorganism is engineered to express or over express a fructose 1,6 bisphosphatase.

The disclosure provides a recombinant microorganism comprising a non-CO₂-evolving pathway that comprises synthesizing acetyl phosphate using a recombinant metabolic pathway that metabolizes methanol, methane, formate, formaldehyde, CO₂, CO, a carbohydrate having the general formula CₙH₂ₙOₙ wherein n=3 to 7, or a sugar phosphate metabolite, with improved carbon yield beyond those obtained by pathways that involve pyruvate decarboxylation. In one embodiment, the microorganism can convert any sugar phosphate to acetyl phosphate with improved yield beyond those obtained by pathways that involve pyruvate decarboxylation. In another embodiment, the sugar phosphate is selected from the group consisting of: sugar phosphates of a triose (G3P, DHAP), an erythrose (E4P), a pentose (R5P, Ru5P, RuBP, X5P), a hexose (F6P, H6P, FBP, G6P), and a sedoheptulose (S7P, SBP). In another embodiment, the sugar phosphates are derived from methanol, methane, CO₂, CO, formaldehyde, formate, glycerol, a carbohydrate having the general formula CₙH₂ₙOₙ, wherein n=3 to 7, or cellulose as a carbon source. In another embodiment, the microorganism is a prokaryote or eukaryote. In another embodiment, the microorganism is yeast. In another embodiment, the microorganism is a prokaryote. In another embodiment, the microorganism is derived from an E. coli microorganism. In another embodiment, the E. coli is engineered to express a phosphoketolase. In another embodiment, the phosphoketolase is Fpk, Xpk or a bifunctional F/Xpk enzyme. In any of the foregoing embodiments, the microorganism is engineered to heterologously expresses one or more of the following enzymes: (a) a phosphoketolase (F/Xpk); (b) a transaldolase (Tal); (c) a transketolase (Tkt); (d) a ribose-5-phosphate isomerase (Rpi); (e) a ribulose-5-phosphate epimerase (Rpe); (f) a triose phosphate isomerase (Tpi); (g) a fructose 1,6 bisphosphate aldolase (Fba); (h) a sedoheptulose bisphosphate aldolase (Sba); (i) a fructose 1,6 bisphosphatase (Fbp); and (j) a sedoheptulose 1,6, bisphosphatase (Sbp). In any of the foregoing embodiments, the microorganism is engineered to express a phosphoketolase derived from Bifidobaceterium adolescentis. In another embodiment, the phosphoketolase comprises a sequence that is at least 49% identical to SEQ ID NO:2 and has phosphoketolase activity. In another embodiment, the microorganism is engineered to express or over express a fructose 1,6 bisphosphatase.

The disclosure also provides a recombinant microorganism comprising a pathway that produces acetyl-phosphate through carbon rearrangement of E4P and metabolism of a carbon source selected from methanol, methane, formate, formaldehyde, CO₂, CO, a carbohydrate (CₙH₂ₙOₙ, n=3-7) or a sugar phosphate. In one embodiment, the microorganism can convert any sugar phosphate to acetyl phosphate with improved yield beyond those obtained by pathways that involve pyruvate decarboxylation. In another embodiment, the sugar phosphate is selected from the group consisting of: sugar phosphates of a triose (G3P, DHAP), an erythrose (E4P), a pentose (R5P, Ru5P, RuBP, X5P), a hexose (F6P, H6P, FBP, G6P), and a sedoheptulose (S7P, SBP). In another embodiment, the sugar phosphates are derived from methanol, methane, CO₂, CO, formaldehyde, formate, glycerol, a carbohydrate having the general formula CₙH₂ₙOₙ, wherein n=3 to 7, or cellulose as a carbon source. In another embodiment, the microorganism is a prokaryote or eukaryote. In another embodiment, the microorganism is yeast. In another embodiment, the microorganism is a prokaryote. In another embodiment, the microorganism is derived from an E. coli microorganism. In another embodiment, the E. coli is engineered to express a phosphoketolase. In another embodiment, the phosphoketolase is Fpk, Xpk or a bifunctional F/Xpk enzyme. In any of the foregoing embodiments, the microorganism is engineered to heterologously expresses one or more of the following enzymes: (a) a phosphoketolase (F/Xpk); (b) a transaldolase (Tal); (c) a transketolase (Tkt); (d) a ribose-5-phosphate isomerase (Rpi); (e) a ribulose-5-phosphate epimerase (Rpe); (f) a triose phosphate isomerase (Tpi); (g) a fructose 1,6 bisphosphate aldolase (Fba); (h) a sedoheptulose bisphosphate aldolase (Sba); (i) a fructose 1,6 bisphosphatase (Fbp); and (j) a sedoheptulose 1,6, bisphosphatase (Sbp). In any of the foregoing embodiments, the microorganism is engineered to express a phosphoketolase derived from Bifidobaceterium adolescentis. In another embodiment, the phosphoketolase comprises a sequence that is at least 49% identical to SEQ ID NO:2 and has phosphoketolase activity. In another embodiment, the microorganism is engineered to express or over express a fructose 1,6 bisphosphatase.

The disclosure also provides a recombinant microorganism expressing enzymes that catalyze the conversion described in (i)-(ix), wherein at least one enzyme or the regulation of at least one enzyme that performs a conversion described in (i)-(ix) is heterologous to the microorganism: (i) the production of acetyl-phosphate and erythrose-4-phosphate (E4P) from fructose-6-phosphate and/or the production of acetyl-phosphate and glyceraldehyde 3-phosphate (G3P) from xylulose 5-phosphate; (ii) the conversion of fructose-6-phosphate and E4P to sedoheptulose 7-phosphate (S7P) and (G3P) or the reverse thereof; (iii) the conversion of S7P and G3P to ribose-5-phosphate and xylulose-5-phosphate or the reverse thereof; (iv) the conversion of ribose-5-phosphate to ribulose-5-phosphate or the reverse thereof; (v) the conversion of ribulose-5-phosphate to xylulose-5-phosphate or the reverse thereof; (vi) the conversion of xylulose-5-phosphate and E4P to fructose-6-phosphate and glyceraldehyde-3-phosphate or the reverse thereof; (vii) the conversion of glyceraldehyde-3-phosphate to dihydroxyacetone phosphate or the reverse thereof; (viii) the conversion of dihydroxyacetone phosphate and glyceraldehyde-3-phosphate to fructose 1,6 bisphosphate or the reverse thereof; and (ix) the conversion of fructose 1,6-biphosphate to fructose-6-phosphate, wherein the microorganism produces acetyl-phosphate or compounds derived from acetyl-phosphate using a carbon source selected from the group consisting of a carbohydrate having the general formula (CₙH₂ₙOₙ, n=3-7), a sugar-phosphate, CO₂, CO, methanol, methane, formate, formaldehyde and any combination thereof. In one embodiment, the microorganism can convert any sugar phosphate to acetyl phosphate with improved yield beyond those obtained by pathways that involve pyruvate decarboxylation. In another embodiment, the sugar phosphate is selected from the group consisting of: sugar phosphates of a triose (G3P, DHAP), an erythrose (E4P), a pentose (R5P, Ru5P, RuBP, X5P), a hexose (F6P, H6P, FBP, G6P), and a sedoheptulose (S7P, SBP). In another embodiment, the sugar phosphates are derived from methanol, methane, CO₂, CO, formaldehyde, formate, glycerol, a carbohydrate having the general formula CₙH₂ₙOₙ, wherein n=3 to 7, or cellulose as a carbon source. In another embodiment, the microorganism is a prokaryote or eukaryote. In another embodiment, the microorganism is yeast. In another embodiment, the microorganism is a prokaryote. In another embodiment, the microorganism is derived from an E. coli microorganism. In another embodiment, the E. coli is engineered to express a phosphoketolase. In another embodiment, the phosphoketolase is Fpk, Xpk or a bifunctional F/Xpk enzyme. In any of the foregoing embodiments, the microorganism is engineered to heterologously expresses one or more of the following enzymes: (a) a phosphoketolase (F/Xpk); (b) a transaldolase (Tal); (c) a transketolase (Tkt); (d) a ribose-5-phosphate isomerase (Rpi); (e) a ribulose-5-phosphate epimerase (Rpe); (f) a triose phosphate isomerase (Tpi); (g) a fructose 1,6 bisphosphate aldolase (Fba); (h) a sedoheptulose bisphosphate aldolase (Sba); (i) a fructose 1,6 bisphosphatase (Fbp); and (j) a sedoheptulose 1,6, bisphosphatase (Sbp). In any of the foregoing embodiments, the microorganism is engineered to express a phosphoketolase derived from Bifidobaceterium adolescentis. In another embodiment, the phosphoketolase comprises a sequence that is at least 49% identical to SEQ ID NO:2 and has phosphoketolase activity. In another embodiment, the microorganism is engineered to express or over express a fructose 1,6 bisphosphatase.

The disclosure also provides a recombinant microorganism comprising a heterologous phosphoketolase or native phosphoketolase under the regulation of a heterologous promoter for the conversion of a sugar phosphate to acetyl-phosphate with improved carbon yield beyond those obtained by pathways that involve pyruvate decarboxylation. In one embodiment, the microorganism uses methanol or methane to produce F6P as a carbon source for the production of acetyl phosphate or acetyl-CoA with improved carbon yield beyond those obtained by pathways that involve pyruvate decarboxylation.

The disclosure also provide a recombinant microorganism comprising a non-CO₂ evolving metabolic pathway for the stoichiometric or improved synthesis of acetyl phosphate with carbon conservation from a carbon substrate using a pathway comprising an enzyme having fructose-6-phosphoketolase (Fpk) activity and/or xylulose-5-phosphoketolase (Xpk) activity. In one embodiment, the microorganism can stoichiometrically convert any sugar phosphate to acetyl phosphate. In another embodiment, the sugar phosphate is selected from the group consisting of: sugar phosphates of a triose (G3P, DHAP), an erythrose (E4P), a pentose (R5P, Ru5P, RuBP, X5P), a hexose (F6P, H6P, FBP, G6P), and a sedoheptulose (S7P, SBP). In yet another embodiment, the sugar phosphates are derived from methanol, methane, CO₂, CO, formaldehyde, formate, glycerol, a carbohydrate having the general formula CₙH₂ₙOₙ, wherein n=3 to 7, or cellulose as a carbon source. In another embodiment, the microorganism is a prokaryote or eukaryote. In yet another embodiment, the microorganism is a yeast. In one embodiment, the microorganism is derived from an E. coli microorganism. In a further embodiment, the E. coli is engineered to express a phosphoketolase. In another embodiment, the phosphoketolase is Fpk, Xpk or a bifunctional F/Xpk enzyme. In any of the foregoing embodiments, the microorganism is engineered to heterologously expresses one or more of the following enzymes: (a) a phosphoketolase (F/Xpk); (b) a transaldolase (Tal); (c) a transketolase (Tkt); (d) a ribose-5-phosphate isomerase (Rpi); (e) a ribulose-5-phosphate epimerase (Rpe); (f) a triose phosphate isomerase (Tpi); (g) a fructose 1,6 bisphosphate aldolase (Fba); (h) a sedoheptulose bisphosphate aldolase (Sba); (i) a fructose 1,6 bisphosphatase (Fbp); and (j) a sedoheptulose 1,6, bisphosphatase (Sbp). In any of the foregoing embodiments, the microorganism is engineered to express a phosphoketolase derived from Bifidobaceterium adolescentis. In another embodiment, the phosphoketolase comprises a sequence that is at least 49% identical to SEQ ID NO:2 and has phosphoketolase activity. In another embodiment, the microorganism is engineered to express or over express a fructose 1,6 bisphosphatase.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-G shows three variations of non-oxidative glycolysis (NOG) converting F6P to 3 molecules of acetyl-phosphate (AcP). (a) NOG involving only fructose 6-phosphate phosphoketolase (Fpk) activity. (b) NOG involving only xylulose 5-phosphate phosphoketolase (Xpk) activity. (c) NOG involving both Fpk and Xpk activities. (d-g) depicts the NOG pathway in other configurations. Other abbreviations are: G6P, glucose 6-phosphate; F6P, fructose 6-phosphate; FBP, fructose 1,6-bisphosphate; E4P, erythrose-4-phosphate; G3P, glyceraldehyde 3-phosphate; DHAP, dihydroxyacetone phosphate; X5P, xylulose 5-phosphate; R5P, ribose 5-phosphate; Ru5P, ribulose 5-phosphate; S7P, sedoheptulose 7-phosphate; Glk, glucokinase; Pgi, phosphoglucose isomerase; Tal, transaldolase, Tkt, transketolase; Rpi, ribose-5-phosphate isomerase; Rpe, ribulose-5-phosphate 3-epimerase; Tpi, triose isomerase; Fba, fructose bisphosphate aldolase; Fbp, fructose-1,6-bisphosphatase.

FIG. 2 depicts the structure of the NOG pathway with possible variations in a linear fashion.

FIG. 3A-B shows the use of NOG in C1 assimilation. (a) Combination of the CBB cycle with NOG would achieve 100% carbon yield, while CBB cycle followed by EMP loses one CO₂ per AcCoA produced, and the carbon yield is 66%. (b) Combination of the RuMP pathway with NOG would achieve 100% carbon yield from methanol to ethanol (EtOH), while RuMP alone loses one CO₂ per EtOH produced, and the carbon yield is 66%. See FIG. 1 and text for abbreviations. Other abbreviations are: Pyr, pyruvate; H6P, 3-hexulose 6-phosphate; Mdh, methanol dehydrogenase; Hps, 3-hexulose-6-phosphate synthase; Phi, 6-phospho 3-hexuloisomerase; Adh, aldehyde/alcohol dehydrogenase.

FIG. 4A-E shows kinetics of NOG converting F6P to AcP. (a) Kinetic simulation of NOG in a batch system using Fpk only revealed that high Fpk activity caused a kinetic trap, resulting in an equimolar distribution of E4P and AcP. (b) Kinetic simulation of NOG using Xpk activity showed no kinetic trapping effect. (c) In vitro conversion of F6P to AcP using eight purified core enzymes, including F/Xpk, Fbp, Fba, Tkt, Tal, Rpi, Rpe, and Tpi. The starting F6P concentration was 10 mM. The triangles are reactions with all eight enzymes present. The squares are reactions with all enzymes except Tal. (d) In vitro conversion of F6P to acetate, determined by high-pressure liquid chromatography (HPLC). Here the addition of Ack and Pfk (to drain the ATP) allowed the complete conversion of acetyl-phosphate to acetate. A similar control with no Tal produced only one third of the possible acetate from F6P. (e) Conversion of three sugar phosphates F6P, R5P, and G3P to near stoichiometric amounts of AcP. Using the same using the core eight enzymes, 10 mM of each substrate was completely converted to AcP whereas a no Tkt control produced roughly a third.

FIG. 5A-D shows in vivo conversion of Xylose to Acetate via NOG. (a) Plasmid pIB4 was created for expressing Bifidobacterium adolescentis fxpk and encoded by E. coli fbp under the control of the synthetic P_{λ}lac01 promoter. (b) Pathways in the engineered E. coli strains for converting xylose to acetate and other competing products (lactate, ethanol, succinate, and formate production). (c) Coupled NADPH enzyme assays confirming that F/Xpk and Fbp are actively expressed using purified enzyme expressed from JCL118. (d) Xylose was converted to acetate and other products under anaerobic conditions. Strain JCL118 produced near theoretical ratios of acetate/xylose.

FIG. 6A-B shows NOG pathways using different starting materials. (a) NOG with C5-phosphate as an input. (b) NOG with C3-phosphate as an input.

FIG. 7 shows the energetics of NOG compared with other glycolytic pathways.

FIG. 8A-C shows a kinetic simulation for NOG from F6P to AcP (Results are shown in FIG. 4a). (a) Reaction pathway simulated. (b) Definition of reactions, (c) ODE's for the system simulation. The kinetic simulation was performed using COPASI.

FIG. 9 show SDS-PAGE gel of HIS-tagged purified enzymes that were expressed and purified.

FIG. 10 shows a series of NADPH-coupled assays was performed to confirm the activity of each protein. These designs were done to independently test the activity in various combinations to determine if any enzyme was limiting. The results confirmed that all the purified enzymes had activity.

FIG. 11 shows expression of F/Xpk and Fbp in JCL118/pIB4. The plasmid pIB4 was made using pZE12 (Shota et. al 2008) as the vector and f/xpk from B. adolescentis and fbp from E. coli (JCL16 gDNA). Lane 2-5 represent crude extract and 6-9 are HIS-tag elutions.

FIG. 12A-B show (a) The Bifid Shunt can produce the highest amount of ATP from glucose (without respiration) at 2.5 ATP/glucose. Glucose is converted into a mixture of lactate and acetate. (b) The original phosphoketolase pathway uses a portion of the ED pathway and oxidizes glucose to a pentose and CO₂ as a waste. The pentose is then degraded into a mixture of EtOH and lactate to remain redox neutral.

FIGS. 13-20 shows various coding sequences for enzymes useful in the methods and compositions of the disclosure (SEQ ID NOs: 1, 3, 5, 7, 9, 11, 13, and 15, respectively).

FIG. 21 shows a diagram of the anaerobic growth rescue system and higher alcohol production in E. coli. In the presence of AdhE, both n-butanol and n-hexanol are produced in E. coli under anaerobic conditions (connected lines). Elimination of AdhE induces cell growth arrest due to the accumulation of NAD+ and acyl-CoA intermediates. To rescue cellular growth, a long-chain acyl-CoA thioesterase (mBACH, dotted line) was introduced, promoting the consumption of NADH and longer-chain acyl-CoA intermediates to produce fatty acids (hexanoic acid). Abbreviations: Fdh, formate dehydrogenase; AtoB, acetyl-CoA acetyltransferase; BktB, β-ketothiolase; Hbd, 3-hydroxy-acyl-CoA dehydrogenase; Crt, crotonase; Ter, trans-enoyl-CoA reductase; AdhE, aldehyde/alcohol dehydrogenase; mBACH, mouse brain acyl-CoA hydrolase.

FIG. 22 shows various applications of the NOG pathway in the production of other chemicals including biodiesels, biofuels, higher alcohols, amino acids from various carbon sources.

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Sugars, acetyl-CoA, acetyl-phosphate (AcP), and acetate all have the same redox state. Theoretically, it should be possible to split glucose into three molecules of these C2 metabolites in a carbon and redox neutral manner. Pathways without excess redox equivalents, would more efficient and could lead to maximal yields. However, no such pathway is known to exist.

The disclosure provides methods and compositions (including cell free systems and recombinant organisms) that provide improved carbon yield compared to a pyruvate decarboxylation process for the production of acetyl-phosphate. By “improved carbon yield” means that the process results in stoichiometric conversion of a starting carbon source to acetyl-phosphate. For example, the methods and compositions of the disclosure can provide a ratio of conversion of Fructose-6-phosphate to acetyl-phosphate that is better than 1:2. In another embodiment, the disclosure provides a carbon utilization that is greater than that of pyruvate decarboxylation (pyruvate decarboxylation has a conversion equal to or less than 1:2). The disclosure provides a non-oxidative glycolytic (NOG) pathway to break down carbohydrates or sugar phosphates into the theoretical maximum amount of two-carbon metabolites without carbon loss. This synthetic pathway contains well-established enzymes found in three distinct pathways: the pentose phosphate pathway (PPP), gluconeogenesis, and the phosphoketolase pathway. The metabolic logic of NOG is analogous to that used in multiple natural pathways: 1) initial investment of a metabolite, which is then regenerated by recycling, 2) reversible ketol-aldol rearrangement, and 3) irreversible reactions serving as driving forces.

It should be recognized that the disclosure describes that pathway as a non-oxidative pathway. The non-oxidative pathway is set forth in FIG. 1. It will be further recognized the oxidative metabolism may occur prior to a sugar phosphate or after production of acetyl-phosphate of FIG. 1.

In the pathways shown (in FIG. 1), fructose 6-phosphate (F6P) is the input molecule. Phosphoketolases (either fructose 6-phosphate phosphoketolase, Fpk, or xylulose 5-phosphate phosphoketolase, Xpk; or a bifunctional F/Xpk) are used to generate acetyl-phosphate (AcP) as an output. The pathway uses investment of erythrose-4-phosphate (E4P), which reacts with F6P to begin a series of reactions involved in non-oxidative carbon rearrangement commonly used in PPP and gluconeogenesis to regenerate E4P. In the process, phosphoketolases and fructose 1,6-bisphosphase (Fbp) provide the irreversible driving forces (FIG. 1A to C). NOG can proceed with Fpk (FIG. 1A), Xpk (FIG. 1B), or bifunctional enzymes that contain both activities (FIG. 1C). Because of the flexibility of NOG, the pathway can proceed with different combinations of Fpk and Xpk, or with different sugar phosphates as the starting molecule (FIG. 6A-B). In all these pathways, NOG converts sugar phosphates to stoichiometric amounts of AcP without carbon loss. AcP can then be converted to acetyl-CoA by acetyltransferase (Pta, Pta variant or homolog thereof), or to acetate by acetate kinase (Ack, Ack variant or homolog thereof). Acetyl-CoA can be converted to alcohols, fatty acids, or other products if additional reducing power is provided. When producing acetate from glucose, NOG splits glucose to three molecules of acetate with a net production of 2 ATP. This pathway is non-oxidative, and involves the largest Gibb's free energy drop compared with EMP to lactate or ethanol and CO₂ (FIG. 7). Acetogens such as Moorella thermoacetica accomplishes carbon conservation by fixing CO₂ emitted from pyruvate via the Wood-Ljungdahl pathway, which contains complex enzymes to overcome significant kinetic or thermodynamic barriers. In contrast, NOG contains no difficult enzymes and is amenable to heterologous expression.

NOG can also be used in conjunction with C1 assimilation pathways that produce acetyl-CoA from pyruvate. When combined with the CBB cycle (FIG. 3A), NOG provides the complete carbon conversion in the synthesis of acetyl-CoA from CBB intermediates such as F6P or glyceraldehyde 3-phosphate (G3P). This combination allows the cell to produce one molecule of acetyl-CoA by fixing two molecules of CO₂, which is a 50% increase in carbon efficiency over the traditional combination of CBB and EMP pathways. In addition, when combined with the RuMP pathway (FIG. 3B), NOG allows the stoichiometric conversion of methanol to form ethanol or butanol. This capability is of particular interest because of the renewed interest in the conversion of C1 compounds to higher carbon chemicals.

Since NOG involves multiple interacting metabolic cycles (FIG. 1A to C), a theoretical simulation was performed to test its feasibility (FIG. 8) using ordinary differential equation (ODE)-based kinetic models. Interestingly, dynamic simulation of the FPK-only NOG (FIG. 1A) showed that having high Fpk activity causes an accumulation of the intermediate E4P. If the activity of Fpk is significantly greater than the rest of the enzymes in a NOG pathway, then all the F6P is trapped as E4P and AcP in an equimolar ratio (FIG. 4A). This kinetic trap is caused by the reduced ability to recycle E4P due to the relatively weak activity of other enzymes. In contrast, when Xpk activity is present (FIGS. 1B and C), even when using extremely high levels of Xpk, no accumulation of any intermediate is seen and the maximum conversion is achieved (FIG. 4B). Such robustness is attributed to the fact that G3P is a “self-generating” intermediate that can form all the other intermediates in the NOG family without any initial investment. Since E4P cannot isomerize and combine with itself (unlike G3P), it is unable to generate other required intermediates to complete the NOG cycle. Thus, if the F6P is degraded too quickly by Fpk, the NOG cycle is split and only one-third of the possible C2 compounds can be produced. In order to reach the maximum conversion of F6P to three molecules of AcP and avoid E4P accumulation, it is preferable, but not necessary, to use Xpk only or dual-function Fpk/Xpk enzymes. Fortunately, most of the reported phosphoketolases have either Xpk or dual Fpk/Xpk activities.

When the model was extended to convert xylose to acetate, the excess ATP produced caused a cofactor imbalance, although in the cell this net production of ATP is beneficial to the cell. This excessive ATP formation may reduce conversion by altering the equilibrium for acetate kinase. By adding a futile ATP-burning cycle using phosphofructokinase, the modeled conversion rate was sped up dramatically due to the regeneration of the ADP cofactor.

In order to prove experimentally the feasibility of this pathway beyond the theoretical, both in vitro and in vivo systems were constructed to demonstrate NOG. Both in vitro and in vivo systems provided a robust and effective metabolic pathway for the production of acetyl-phosphate. Thus, the disclosure provides both a cell-free (in vitro) pathway and a recombinant microorganism pathway for the production of acetyl-phosphate.

The disclosure provides an in vitro method of producing acetyl-phosphate, acetyl-CoA and chemicals and biofuels that use acetyl-CoA as a substrate. In this embodiment, of the disclosure cell-free preparations can be made through, for example, three methods. In one embodiment, the enzymes of the NOG pathway, as described more fully below, are purchased and mixed in a suitable buffer and a suitable substrate is added and incubated under conditions suitable for acetyl-phosphate production. In another embodiment, one or more polynucleotides encoding one or more enzymes of the NOG pathway are cloned into one or more microorganism under conditions whereby the enzymes are expressed. Subsequently the cells are lysed and the lysed preparation comprising the one or more enzymes derived from the cell are combined with a suitable buffer and substrate (and one or more additional enzymes of the NOG pathway) to produced acetyl-phosphate from the substrate. Alternatively, the enzymes can be isolated from the lysed preparations and then recombined in an appropriate buffer. In yet another embodiment, a combination of purchased enzymes and express enzymes are used to provide a NOG pathway in an appropriate buffer.

For example, to construct an in vitro system, all the NOG enzymes were acquired commercially or purified by affinity chromatography (FIG. 9), tested for activity (FIG. 10), and mixed together in a properly selected reaction buffer. The system was ATP- and redox-independent and comprised eight enzymes: Fpk/Xpk, Fbp, fructose bisphosphate aldolase (Fba), triose phosphate isomerase (Tpi), ribulose-5-phosphate 3-epimerase (Rpe), ribose-5-phosphate isomerase (Rpi), transketolase (Tkt), and transaldolase (Tal). Acetyl-phosphate concentration was measured using an end-point colorimetric hydroxamate method. Using this in vitro system an initial 10 mM amount of F6P was completely converted to stoichiometric amounts of AcP (within error) at room temperature after 1.5 hours (FIG. 4C). As a control, when no Tal was added, only one-third of the AcP was produced (FIG. 4C).

To extend the production further to acetate, Ack was added to the in vitro NOG system. On the basis of the simulation discussed above, phosphofructokinase was also added to maintain ATP-balance. Since the ADP (the substrate for acetate kinase) is regenerated, only a catalytic amount (20 μM) was necessary. Acetate concentration monitored by HPLC showed maximum conversion (FIG. 4D), which was three-times higher than that produced by the control with no Tal added. Without the complete NOG, F6P was converted to equilimolar amounts of E4P and acetate in a linear pathway. Since the core portion of NOG can convert any sugar phosphate (e.g., triose to sedoheptulose) to stoichiometric amounts of AcP, similar in vitro systems were tested on ribose-5-phosphate and G3P. These two compounds produced nearly theoretical amounts of acetyl-phosphate at 2.3 and 1.6 mM of AcP per mM of substrate, respectively (FIG. 4E).

After demonstrating in vitro feasibility of NOG, an in vivo model was generated as described more fully below. Using the foregoing enzymes a biosynthetic pathway was engineered into a microorganism to obtain a recombinant microorganism.

The disclosure provides recombinant organisms comprising metabolically engineered biosynthetic pathways that comprise a non-CO₂ evolving pathway for the production of acetyl-phosphate, acetyl-CoA and/or products derived therefrom.

In one embodiment, the disclosure provides a recombinant microorganism comprising elevated expression of at least one target enzyme as compared to a parental microorganism or encodes an enzyme not found in the parental organism. In another or further embodiment, the microorganism comprises a reduction, disruption or knockout of at least one gene encoding an enzyme that competes with a metabolite necessary for the production of a desired metabolite or which produces an unwanted product. The recombinant microorganism produces at least one metabolite involved in a biosynthetic pathway for the production of, for example, acetyl-phosphate and/or acetyl-CoA. In general, the recombinant microorganisms comprises at least one recombinant metabolic pathway that comprises a target enzyme and may further include a reduction in activity or expression of an enzyme in a competitive biosynthetic pathway. The pathway acts to modify a substrate or metabolic intermediate in the production of, for example, acetyl-phosphate and/or acetyl-CoA. The target enzyme is encoded by, and expressed from, a polynucleotide derived from a suitable biological source. In some embodiments, the polynucleotide comprises a gene derived from a bacterial or yeast source and recombinantly engineered into the microorganism of the disclosure.

As used herein, an “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product. The disclosure provides recombinant microorganism having a metabolically engineered pathway for the production of a desired product or intermediate.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite. In an illustrative embodiment, the introduction of genetic material into a parental microorganism results in a new or modified ability to produce acetyl-phosphate and/or acetyl-CoA through a non-CO₂ evolving and/or non-oxidative pathway for optimal carbon utilization. The genetic material introduced into the parental microorganism contains gene(s), or parts of gene(s), coding for one or more of the enzymes involved in a biosynthetic pathway for the production of acetyl-phosphate and/or acetyl-CoA, and may also include additional elements for the expression and/or regulation of expression of these genes, e.g. promoter sequences.

An engineered or modified microorganism can also include in the alternative or in addition to the introduction of a genetic material into a host or parental microorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new or greater quantities of an intracellular metabolite, improve the flux of a metabolite down a desired pathway, and/or reduce the production of undesirable by-products).

An “enzyme” means any substance, typically composed wholly or largely of amino acids making up a protein or polypeptide that catalyzes or promotes, more or less specifically, one or more chemical or biochemical reactions.

As used herein, the term “metabolically engineered” or “metabolic engineering” involves rational pathway design and assembly of biosynthetic genes, genes associated with operons, and control elements of such polynucleotides, for the production of a desired metabolite, such as an acetyl-phosphate and/or acetyl-CoA, higher alcohols or other chemical, in a microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

A “metabolite” refers to any substance produced by metabolism or a substance necessary for or taking part in a particular metabolic process that gives rise to a desired metabolite, chemical, alcohol or ketone. A metabolite can be an organic compound that is a starting material (e.g., a carbohydrate, a sugar phosphate, pyruvate etc.), an intermediate in (e.g., acetyl-coA), or an end product (e.g., 1-butanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

A “native” or “wild-type” protein, enzyme, polynucleotide, gene, or cell, means a protein, enzyme, polynucleotide, gene, or cell that occurs in nature.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that serves as the “parent” for further engineering.

For example, a wild-type microorganism can be genetically modified to express or over express a first target enzyme such as a phosphoketolase. This microorganism can act as a parental microorganism in the generation of a microorganism modified to express or over-express a second target enzyme e.g., a transaldolase. In turn, that microorganism can be modified to express or over express e.g., a transketolase and a ribose-5 phosphate isomerase, which can be further modified to express or over express a third target enzyme, e.g., a ribulose-5-phosphate epimerase.

Accordingly, a parental microorganism functions as a reference cell for successive genetic modification events. Each modification event can be accomplished by introducing one or more nucleic acid molecules in to the reference cell. The introduction facilitates the expression or over-expression of one or more target enzyme or the reduction or elimination of one or more target enzymes. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme in to a parental microorganism.

A “protein” or “polypeptide”, which terms are used interchangeably herein, comprises one or more chains of chemical building blocks called amino acids that are linked together by chemical bonds called peptide bonds. A protein or polypeptide can function as an enzyme.

Polynucleotides that encode enzymes useful for generating metabolites (e.g., enzymes such as phosphoketolase, transaldolase, transketolase, ribose-5-phosphate isomerase, ribulose-5-phosphate epimerase, triose phosphate isomerase, fructose 1,6-bisphosphase aldolase, fructose 1,6 bisphosphatase) including homologs, variants, fragments, related fusion proteins, or functional equivalents thereof, are used in recombinant nucleic acid molecules that direct the expression of such polypeptides in appropriate host cells, such as bacterial or yeast cells. FIGS. 13-20 provide exemplary polynucleotide sequences encoding polypeptides useful in the methods described herein. It is understood that the addition of sequences which do not alter the encoded activity of a nucleic acid molecule, such as the addition of a non-functional or non-coding sequence, is a conservative variation of the basic nucleic acid.

It is understood that a polynucleotide described above include “genes” and that the nucleic acid molecules described above include “vectors” or “plasmids.” For example, a polynucleotide encoding a phosphoketolase can comprise an Fpk gene or homolog thereof, or an Xpk gene or homolog thereof, or a bifunctional F/Xpk gene or homolog thereof. Accordingly, the term “gene”, also called a “structural gene” refers to a polynucleotide that codes for a particular polypeptide comprising a sequence of amino acids, which comprise all or part of one or more proteins or enzymes, and may include regulatory (non-transcribed) DNA sequences, such as promoter region or expression control elements, which determine, for example, the conditions under which the gene is expressed. The transcribed region of the gene may include untranslated regions, including introns, 5′-untranslated region (UTR), and 3′-UTR, as well as the coding sequence.

The term “polynucleotide,” “nucleic acid” or “recombinant nucleic acid” refers to polynucleotides such as deoxyribonucleic acid (DNA), and, where appropriate, ribonucleic acid (RNA).

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate preferred embodiments of the disclosure.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form.

A polynucleotide of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated polynucleotide molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitution, in some positions it is preferable to make conservative amino acid substitutions.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

The term “recombinant microorganism” and “recombinant host cell” are used interchangeably herein and refer to microorganisms that have been genetically modified to express or over-express endogenous polynucleotides, or to express non-endogenous sequences, such as those included in a vector. The polynucleotide generally encodes a target enzyme involved in a metabolic pathway for producing a desired metabolite as described above, but may also include protein factors necessary for regulation or activity or transcription. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, but also intermediate and end product metabolites used in a pathway associated with a metabolically engineered microorganism as described herein. With respect to the NOG pathway described herein, a starting material can be any suitable carbon source including, but not limited to, glucose, fructose or other biomass sugars, methanol, methane, glycerol, CO₂ etc. These starting materials may be metabolized to a suitable sugar phosphate that enters the NOG pathway as set forth in FIG. 1.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of a gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

The disclosure provides methods for the heterologous expression of one or more of the biosynthetic genes or polynucleotides involved in acetyl-phosphate synthesis, acetyl-CoA biosynthesis or other metabolites derived therefrom and recombinant DNA expression vectors useful in the method. Thus, included within the scope of the disclosure are recombinant expression vectors that include such nucleic acids.

Recombinant microorganisms provided herein can express a plurality of target enzymes involved in pathways for the production of acetyl-phosphate, acetyl-CoA or other metabolites derived therefrom from a suitable carbon substrate such as, for example, glucose, fructose or other biomass sugars, methanol, methane, glycerol, CO₂ and the like. The carbon source can be metabolized to, for example, a desirable sugar phosphate that then feeds into the NOG pathway of the disclosure. A “biomass derived sugar” includes, but is not limited to, molecules such as glucose, sucrose, mannose, xylose, and arabinose. The term biomass derived sugar encompasses suitable carbon substrates of 1 to 7 carbons ordinarily used by microorganisms, such as 3-7 carbon sugars, including but not limited to glucose, lactose, sorbose, fructose, idose, galactose and mannose all in either D or L form, or a combination of 3-7 carbon sugars, such as glucose and fructose, and/or 6 carbon sugar acids including, but not limited to, 2-keto-L-gulonic acid, idonic acid (IA), gluconic acid (GA), 6-phosphogluconate, 2-keto-D-gluconic acid (2 KDG), 5-keto-D-gluconic acid, 2-ketogluconatephosphate, 2,5-diketo-L-gulonic acid, 2,3-L-diketogulonic acid, dehydroascorbic acid, erythorbic acid (EA) and D-mannonic acid.

Cellulosic and lignocellulosic feedstocks and wastes, such as agricultural residues, wood, forestry wastes, sludge from paper manufacture, and municipal and industrial solid wastes, provide a potentially large renewable feedstock for the production of chemicals, plastics, fuels and feeds. Cellulosic and lignocellulosic feedstocks and wastes, composed of carbohydrate polymers comprising cellulose, hemicellulose, and lignin can be generally treated by a variety of chemical, mechanical and enzymatic means to release primarily hexose and pentose sugars. These sugars can then be “fed” into the NOG pathway described herein, which can then be fermented to useful products including 1-butanol, isobutanol, ethanol, 2-pentanone, octanol and the like.

The disclosure demonstrates that the expression or over expression of one or more heterologous polynucleotide or over-expression of one or more native polynucleotides encoding (i) a polypeptide that catalyzes the production of acetyl-phosphate and erythrose-4-phosphate (E4P) from Fructose-6-phosphate; (ii) a polypeptide that catalyzes the conversion of fructose-6-phosphate and E4P to sedoheptulose 7-phosphate (S7P); (iii) a polypeptide the catalyzes the conversion of S7P to ribose-5-phosphate and xylulose-5-phosphate; (iv) a polypeptide that catalyzes the conversion of ribose-5-phosphate to ribulose-5-phosphate; (v) a polypeptide the catalyzes the conversion of ribulose-5-phosphate to xylulose-5-phosphate; (vi) a polypeptide that converts xylulose-5-phosphate and E4P to fructose-6-phosphate and glyceraldehyde-3-phosphate; (vii) a polypeptide that converts glyceraldehyde-3-phosphate to dihydroxyacetone phosphate; (viii) a polypeptide that converts dihydroxyacetone phosphate and glyceraldehyde-3-phosphate to fructose 1,6 bisphosphate and (vii) a polypeptide that converts fructose 1,6-bisphosphate to fructose-6-phosphate. For example, the disclosure demonstrates that with expression of the heterologous an Fpk/Xpk genes in Escherichia (e.g., E. coli) the production of acetyl-phosphate, acetyl-CoA or other metabolites derived therefrom can be obtained.

Microorganisms provided herein are modified to produce metabolites in quantities and utilize carbon sources more effectively compared to a parental microorganism. In particular, the recombinant microorganism comprises a metabolic pathway for the production of acetyl-phosphate that conserves carbon. By “conserves carbon” is meant that the metabolic pathway that converts a sugar phosphate to acetyl-phosphate has a minimal or no loss of carbon from the starting sugar phosphate to the acetyl-phosphate. For example, the recombinant microorganism produces a stoichiometrically conserved amount of carbon product from the same number of carbons in the input sugar phosphate (e.g., 1 Fructose-6-P produces 3 acetyl-phosphates).

Accordingly, the disclosure provides a recombinant microorganisms that produce acetyl-phosphate, acetyl-CoA or other metabolites derived therefrom and includes the expression or elevated expression of target enzymes such as a phosphoketolase (e.g., Fpk, Xpk, or Fpk/Xpk), a transaldolase (e.g., Tal), a transketolase (e.g., Tkt), ribose-5-phosphate isomerase (e.g., Rpi), a ribulose-5-phosphate epimerase (e.g., Rpe), a triose phosphate isomerase (e.g., Tpi), a fructose 1,6 bisphosphate aldolase (e.g., Fba), a fructose 1,6 bisphosphatase (e.g., Fbp), or any combination thereof, as compared to a parental microorganism. In addition, the microorganism may include a disruption, deletion or knockout of expression of an alcohol/acetaldehyde dehydrogenase that preferentially uses acetyl-coA as a substrate (e.g. adhE gene), as compared to a parental microorganism. In some embodiments, further knockouts may include knockouts in a lactate dehydrogenase (e.g., ldh) and frdBC. It will be recognized that organism that inherently have one or more (but not all) of the foregoing enzymes can be utilized as a parental organism. As described more fully below, a microorganism of the disclosure comprising one or more recombinant genes encoding one or more enzymes above, may further include additional enzymes that extend the acetyl-phosphate product to acetyl-CoA, which can then be extended to produce, for example, butanol, isobutanol, 2-pentanone and the like.

Accordingly, a recombinant microorganism provided herein includes the elevated expression of at least one target enzyme, such as FpK, Xpk, or F/Xpk. In other embodiments, a recombinant microorganism can express a plurality of target enzymes involved in a pathway to produce acetyl-phosphate, acetyl-CoA or other metabolites derived therefrom as depicted in FIG. 1 from a sugar-phosphate intermediate. In one embodiment, the recombinant microorganism comprises expression of a heterologous or over expression of an endogenous enzyme selected from a phosphoketolase and either a sedoheptulose bisphosphatase or a fructose bisphosphatase. In another embodiment, when the microorganism expresses or overexpress a sedoheptulose bisphosphatase (sbp) or a sedoheptulose bisphosphate aldolase the microorganism does not express a transaldolase.

As previously noted, the target enzymes described throughout this disclosure generally produce metabolites. In addition, the target enzymes described throughout this disclosure are encoded by polynucleotides. For example, a fructose-6-phosphoketolase can be encoded by an Fpk gene, polynucleotide or homolog thereof. The Fpk gene can be derived from any biologic source that provides a suitable nucleic acid sequence encoding a suitable enzyme having fructose-6-phosphoketolase activity.

Accordingly, in one embodiment, a recombinant microorganism provided herein includes expression of a fructose-6-phosphoketolase (Fpk) as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes acetyl-phosphate and E4P from fructose-6-phosphate. The fructose-6-phosphoketolase can be encoded by an Fpk gene, polynucleotide or homolog thereof. The Fpk gene or polynucleotide can be derived from Bifidobacterium adolescentis.

Phosphoketolase enzymes (F/Xpk) catalyze the formation of acetyl-phosphate and glyceraldehyde 3-phosphate or erythrose-4-phosphate from xylulose 5-phosphate or fructose 6-phosphate, respectively. For example, the Bifidobacterium adolescentis Fpk and Xpk genes or homologs thereof can be used in the methods of the disclosure.

In addition to the foregoing, the terms “phosphoketolase” or “F/Xpk” refer to proteins that are capable of catalyzing the formation of acetyl-phosphate and glyceraldehyde 3-phosphate or erythrose-4-phosphate from xylulose 5-phosphate or fructose 6-phosphate, respectively, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:2. Additional homologs include: Gardnerella vaginalis 409-05 ref|YP_{—}003373859.1| having 91% identity to SEQ ID NO:2; Bifidobacterium breve ref|ZP_{—}06595931.1| having 89% to SEQ ID NO:2; Cellulomonas fimi ATCC 484 YP_{—}004452609.1 having 55% to SEQ ID NO:2; Methylomonas methanica YP_{—}004515101.1 having 50% identity to SEQ ID NO:2; and Thermosynechococcus elongatus BP-1] NP_{—}681976.1 having 49% identity to SEQ ID NO:2. The sequences associated with the foregoing accession numbers are incorporated herein by reference.

In another embodiment, a recombinant microorganism provided herein includes elevated expression of a fructose 1,6 bisphosphatase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes a fructose 6-phosphate from a substrate that includes fructose 1,6 bisphosphate. The fructose 1,6 bisphosphatase can be encoded by an Fbp gene, polynucleotide or homolog thereof. The Fbp gene can be derived from various microorganisms including E. coli.

In addition to the foregoing, the terms “fructose 1,6 bisphosphatease” or “Fbp” refer to proteins that are capable of catalyzing the formation of fructose-6-phosphate from fructose-1,6-bisphosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:4. Additional homologs include: Shigella flexneri K-272 ZP_{—}12359472.1 having 99% identity to SEQ ID NO:4; Pantoea agglomerans IG1 ZP_{—}09512587.1 having 85% identity to SEQ ID NO:4; Vibrio cholerae V52 ZP_{—}01680565.1 having 77% identity to SEQ ID NO:4; Aeromonas aquariorum AAK1 ZP_{—}11385413.1 having 72% identity to SEQ ID NO:2; and Desulfovibrio desulfuricans YP_{—}002479779.1 having 50% identity to SEQ ID NO:4. The sequences associated with the foregoing accession numbers are incorporated herein by reference.

In another embodiment, a recombinant microorganism provided herein includes elevated expression of ribulose-5-phosphate epimerase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes xylulose 5-phosphate from a substrate that includes ribulose 5-phosphate. The ribulose-5-phosphate epimerase can be encoded by an Rpe gene, polynucleotide or homolog thereof. The Rpe gene or polynucleotide can be derived from various microorganisms including E. coli.

In addition to the foregoing, the terms “ribulose 5-phosphate epimerase” or “Rpe” refer to proteins that are capable of catalyzing the formation of xylulose 5-phosphate from ribulose 5-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:6. Additional homologs include: Shigella boydii ATCC 9905 ZP_{—}11645297.1 having 99% identity to SEQ ID NO:6; Shewanella loihica PV-4 YP_{—}001092350.1 having 87% identity to SEQ ID NO:6; Nitrosococcus halophilus Nc4 YP_{—}003526253.1 having 75% identity to SEQ ID NO:6; Ralstonia eutropha JMP134 having 72% identity to SEQ ID NO:6; and Synechococcus sp. CC9605 YP_{—}381562.1 having 51% identity to SEQ ID NO:6. The sequences associated with the foregoing accession numbers are incorporated herein by reference.

In another embodiment, a recombinant microorganism provided herein includes elevated expression of ribose-5-phosphate isomerase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes ribulose-5-phosphate from a substrate that includes ribose-5-phosphate. The ribose-5-phosphate isomerase can be encoded by an Rpi gene, polynucleotide or homolog thereof. The Rpi gene or polynucleotide can be derived from various microorganisms including E. coli.

In addition to the foregoing, the terms “ribose-5-phosphate isomerase” or “Rpi” refer to proteins that are capable of catalyzing the formation of ribulose-5-phosphate from ribose 5-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:8. Additional homologs include: Vibrio sinaloensis DSM 21326 ZP_{—}08101051.1 having 74% identity to SEQ ID NO:8; Aeromonas media WS ZP_{—}15944363.1 having 72% identity to SEQ ID NO:8; Thermosynechococcus elongatus BP-1 having 48% identity to SEQ ID NO:8; Lactobacillus suebicus KCTC 3549 ZP_{—}09450605.1 having 42% identity to SEQ ID NO:8; and Homo sapiens AAK95569.1 having 37% identity to SEQ ID NO:8. The sequences associated with the foregoing accession numbers are incorporated herein by reference.

In another embodiment, a recombinant microorganism provided herein includes elevated expression of transaldolase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes sedoheptulose-7-phosphate from a substrate that includes erythrose-4-phosphate and fructose-6-phosphate. The transaldolase can be encoded by a Tal gene, polynucleotide or homolog thereof. The Tal gene or polynucleotide can be derived from various microorganisms including E. coli.

In addition to the foregoing, the terms “transaldolase” or “Tal” refer to proteins that are capable of catalyzing the formation of sedoheptulose-7-phosphate from erythrose-4-phosphate and fructose-6-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:10. Additional homologs include: Bifidobacterium breve DSM 20213 ZP_{—}06596167.1 having 30% identity to SEQ ID NO:10; Homo sapiens AAC51151.1 having 67% identity to SEQ ID NO:10; Cyanothece sp. CCY0110 ZP_{—}01731137.1 having 57% identity to SEQ ID NO:10; Ralstonia eutropha JMP134 YP_{—}296277.2 having 57% identity to SEQ ID NO:10; and Bacillus subtilis BEST7613 NP_{—}440132.1 having 59% identity to SEQ ID NO:10. The sequences associated with the foregoing accession numbers are incorporated herein by reference.

In another embodiment, a recombinant microorganism provided herein includes elevated expression of transketolase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes (i) ribose-5-phosphate and xylulose-5-phosphate from sedoheptulose-7-phosphate and glyceraldehyde-3-phosphate; and/or (ii) glyceraldehyde-3-phosphate and fructose-6-phosphate from xylulose-5-phosphate and erythrose-4-phosphate. The transketolase can be encoded by a Tkt gene, polynucleotide or homolog thereof. The Tkt gene or polynucleotide can be derived from various microorganisms including E. coli.

In addition to the foregoing, the terms “transketolase” or “Tkt” refer to proteins that are capable of catalyzing the formation of (i) ribose-5-phosphate and xylulose-5-phosphate from sedoheptulose-7-phosphate and glyceraldehyde-3-phosphate; and/or (ii) glyceraldehyde-3-phosphate and fructose-6-phosphate from xylulose-5-phosphate and erythrose-4-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:12. Additional homologs include: Neisseria meningitidis M13399 ZP_{—}11612112.1 having 65% identity to SEQ ID NO:12; Bifidobacterium breve DSM 20213 ZP_{—}06596168.1 having 41% identity to SEQ ID NO:12; Ralstonia eutropha JMP134 YP_{—}297046.1 having 66% identity to SEQ ID NO:12; Synechococcus elongatus PCC 6301 YP_{—}171693.1 having 56% identity to SEQ ID NO:12; and Bacillus subtilis BEST7613 NP_{—}440630.1 having 54% identity to SEQ ID NO:12. The sequences associated with the foregoing accession numbers are incorporated herein by reference.

In another embodiment, a recombinant microorganism provided herein includes elevated expression of a triose phosphate isomerase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes dihydroxyacetone phosphate from glyceraldehyde-3-phosphate. The triose phosphate isomerase can be encoded by a Tpi gene, polynucleotide or homolog thereof. The Tpi gene or polynucleotide can be derived from various microorganisms including E. coli.

In addition to the foregoing, the terms “triose phosphate isomerase” or “Tpi” refer to proteins that are capable of catalyzing the formation of dihydroxyacetone phosphate from glyceraldehyde-3-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:14. Additional homologs include: Rattus norvegicus AAA42278.1 having 45% identity to SEQ ID NO:14; Homo sapiens AAH17917.1 having 45% identity to SEQ ID NO:14; Bacillus subtilis BEST7613 NP_{—}391272.1 having 40% identity to SEQ ID NO:14; Synechococcus elongatus PCC 6301 YP_{—}171000.1 having 40% identity to SEQ ID NO:14; and Salmonella enterica subsp. enterica serovar Typhi str. AG3 ZP_{—}06540375.1 having 98% identity to SEQ ID NO:14. The sequences associated with the foregoing accession numbers are incorporated herein by reference.

In another embodiment, a recombinant microorganism provided herein includes elevated expression of a fructose 1,6 bisphosphate aldolase as compared to a parental microorganism. This expression may be combined with the expression or over-expression with other enzymes in the metabolic pathway for the production of acetyl-phosphate, acetyl-CoA or other metabolites derived therefrom as described herein above and below. The recombinant microorganism produces a metabolite that includes fructose 1,6-bisphosphate from a substrate that includes dihydroxyacetone phosphate and glyceraldehyde-3-phosphate. The fructose 1,6 bisphosphate aldolase can be encoded by a Fba gene, polynucleotide or homolog thereof. The Fba gene or polynucleotide can be derived from various microorganisms including E. coli.

In addition to the foregoing, the terms “fructose 1,6 bisphosphate aldolase” or “Fba” refer to proteins that are capable of catalyzing the formation of fructose 1,6-bisphosphate from a substrate that includes dihydroxyacetone phosphate and glyceraldehyde-3-phosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:16. Additional homologs include: Synechococcus elongatus PCC 6301 YP_{—}170823.1 having 26% identity to SEQ ID NO:16; Vibrio nigripulchritudo ATCC 27043 ZP_{—}08732298.1 having 80% identity to SEQ ID NO:16; Methylomicrobium album BG8 ZP_{—}09865128.1 having 76% identity to SEQ ID NO:16; Pseudomonas fluorescens Pf0-1 YP_{—}350990.1 having 25% identity to SEQ ID NO:16; and Methylobacterium nodulans ORS 2060 YP_{—}002502325.1 having 24% identity to SEQ ID NO:16. The sequences associated with the foregoing accession numbers are incorporated herein by reference.

In yet another embodiment, a recombinant microorganism provided herein includes elevated expression of a crotonyl-CoA reductase as compared to a parental microorganism. This expression may be combined with the expression or over-expression with other enzymes in the metabolic pathway for the production of n-butanol, isobutanol, butyryl-coA and/or acetone. The microorganism produces a metabolite that includes butyryl-CoA from a substrate that includes crotonyl-CoA. The crotonyl-CoA reductase can be encoded by a ccr gene, polynucleotide or homolog thereof. The ccr gene or polynucleotide can be derived from the genus Streptomyces. Alternatively, or in addition to, the microorganism provided herein includes elevated expression of a trans-2-hexenoyl-CoA reductase as compared to a parental microorganism. The microorganism produces a metabolite that includes butyryl-CoA from a substrate that includes crotonyl-CoA. The trans-2-hexenoyl-CoA reductase can also convert trans-2-hexenoyl-CoA to hexanoyl-CoA. The trans-2-hexenoyl-CoA reductase can be encoded by a ter gene, polynucleotide or homolog thereof. The ter gene or polynucleotide can be derived from the genus Euglena. The ter gene or polynucleotide can be derived from Treponema denticola. The enzyme from Euglena gracilis acts on crotonoyl-CoA and, more slowly, on trans-hex-2-enoyl-CoA and trans-oct-2-enoyl-CoA.

Trans-2-enoyl-CoA reductase or TER is a protein that is capable of catalyzing the conversion of crotonyl-CoA to butyryl-CoA, and trans-2-hexenoyl-CoA to hexanoyl-CoA. In certain embodiments, the recombinant microorganism expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (see, e.g., U.S. Pat. Appl. 2007/0022497 to Cirpus et al.; and Hoffmeister et al., J. Biol. Chem., 280:4329-4338, 2005, both of which are incorporated herein by reference in their entirety). A truncated cDNA of the E. gracilis gene has been functionally expressed in E. coli.

TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V. angustum, V. cholerae, V. alginolyticus, V. parahaemolyticus, V. vulnificus, V. fischeri, V. splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X. oryzae, X. campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. ambifaria, B. vietnamensis, B. multivorans, B. dolosa, Methylbacillus spp. including, but not limited to, M. flagellatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X. fastidiosa, Reinekea spp., Colweffia spp. including, but not limited to, C. psychrerythraea, Yersinia spp. including, but not limited to, Y. pestis, Y. pseudotuberculosis, Methylobacillus spp. including, but not limited to, M. flagellatus, Cytophaga spp. including, but not limited to, C. hutchinsonii, Flavobacterium spp. including, but not limited to, F. johnsoniae, Microscilla spp. including, but not limited to, M. marina, Polaribacter spp. including, but not limited to, P. irgensii, Clostridium spp. including, but not limited to, C. acetobutylicum, C. beijerenckii, C. cellulolyticum, Coxiella spp. including, but not limited to, C. burnetii.

In addition to the foregoing, the terms “trans-2-enoyl-CoA reductase” or “TER” refer to proteins that are capable of catalyzing the conversion of crotonyl-CoA to butyryl-CoA, or trans-2-hexenoyl-CoA to hexanoyl-CoA and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to either or both of the truncated E. gracilis TER or the full length A. hydrophila TER.

In yet another embodiment, a recombinant microorganism provided herein includes elevated expression of a butyryl-CoA dehydrogenase as compared to a parental microorganism. This expression may be combined with the expression or over-expression with other enzymes in the metabolic pathway for the production of 1-butanol, isobutanol, acetone, octanol, hexanol, 2-pentanone, and butyryl-coA as described herein above and below. The recombinant microorganism produces a metabolite that includes butyryl-CoA from a substrate that includes crotonyl-CoA. The butyryl-CoA dehydrogenase can be encoded by a bcd gene, polynucleotide or homolog thereof. The bcd gene, polynucleotide can be derived from Clostridium acetobutylicum, Mycobacterium tuberculosis, or Megasphaera elsdenii.

In another embodiment, a recombinant microorganism provided herein includes expression or elevated expression of an acetyl-CoA acetyltransferase as compared to a parental microorganism. The microorganism produces a metabolite that includes acetoacetyl-CoA from a substrate that includes acetyl-CoA. The acetyl-CoA acetyltransferase can be encoded by a thlA gene, polynucleotide or homolog thereof. The thlA gene or polynucleotide can be derived from the genus Clostridium.

Pyruvate-formate lyase (Formate acetlytransferase) is an enzyme that catalyzes the conversion of pyruvate to acetyl-coA and formate. It is induced by pfl-activating enzyme under anaerobic conditions by generation of an organic free radical and decreases significantly during phosphate limitation. Formate acetlytransferase is encoded in E. coli by pflB. PFLB homologs and variants are known. For examples, such homologs and variants include, for example, Formate acetyltransferase 1 (Pyruvate formate-lyase 1) gi|129879|sp|P09373.2|PFLB_ECOLI(129879); formate acetyltransferase 1 (Yersinia pestis CO92) gi|16121663|ref|NP_{—}404976.1|(16121663); formate acetyltransferase 1 (Yersinia pseudotuberculosis IP 32953) gi|51595748|ref|YP_{—}069939.1|(51595748); formate acetyltransferase 1 (Yersinia pestis biovar Microtus str. 91001) gi|45441037|ref|NP_{—}992576.1|(45441037); formate acetyltransferase 1 (Yersinia pestis CO92) gi|1153471421|mb|CAL20035.1|(115347142); formate acetyltransferase 1 (Yersinia pestis biovar Microtus str. 91001) gi|45435896|gb|AAS61453.1| (45435896); formate acetyltransferase 1 (Yersinia pseudotuberculosis IP 32953) gi|51589030|emb|CAH20648.1|(51589030); formate acetyltransferase 1 (Salmonella enterica subsp. enterica serovar Typhi str. CT18) gi|16759843|ref|NP_{—}455460.1|(16759843); formate acetyltransferase 1 (Salmonella enterica subsp. enterica serovar Paratyphi A str. ATCC 9150) gi|56413977|ref|YP_{—}151052.1|(56413977); formate acetyltransferase 1 (Salmonella enterica subsp. enterica serovar Typhi) gi|16502136|emb|CAD05373.1|(16502136); formate acetyltransferase 1 (Salmonella enterica subsp. enterica serovar Paratyphi A str. ATCC 9150) gi|56128234|gb|AAV77740.1|(56128234); formate acetyltransferase 1 (Shigella dysenteriae Sd197) gi|82777577|ref|YP_{—}403926.1| (82777577); formate acetyltransferase 1 (Shigella flexneri 2a str. 2457T) gi|30062438|ref|NP_{—}836609.1|(30062438); formate acetyltransferase 1 (Shigella flexneri 2a str. 2457T) gi|30040684|gb|AAP16415.1|(30040684); formate acetyltransferase 1 (Shigella flexneri 5 str. 8401) gi|110614459|gb|ABF03126.1| (110614459); formate acetyltransferase 1 (Shigella dysenteriae Sd197) gi|81241725|gb|ABB62435.1| (81241725); formate acetyltransferase 1 (Escherichia coli O157:H7 EDL933) gi|12514066|gb|AAG55388.1|AE005279_{—}8(12514066); formate acetyltransferase 1 (Yersinia pestis KIM) gi|22126668|ref|NP_{—}670091.1|(22126668); formate acetyltransferase 1 (Streptococcus agalactiae A909) gi|76787667|ref|YP_{—}330335.1|(76787667); formate acetyltransferase 1 (Yersinia pestis KIM) gi|21959683|gb|AAM86342.1|AE013882_{—}3(21959683); formate acetyltransferase 1 (Streptococcus agalactiae A909) gi|76562724|gb|ABA45308.1|(76562724); formate acetyltransferase 1 (Yersinia enterocolitica subsp. enterocolitica 8081) gi|123441844|ref|YP_{—}001005827.1|(123441844); formate acetyltransferase 1 (Shigella flexneri 5 str. 8401) gi|110804911|ref|YP_{—}688431.1|(110804911); formate acetyltransferase 1 (Escherichia coli UTI89) gi|91210004|ref|YP_{—}539990.1|(91210004); formate acetyltransferase 1 (Shigella boydii Sb227) gi|82544641|ref|YP_{—}408588.1| (82544641); formate acetyltransferase 1 (Shigella sonnei Ss046) gi|74311459|ref|YP_{—}309878.1|(74311459); formate acetyltransferase 1 (Klebsiella pneumoniae subsp. pneumoniae MGH 78578) gi|152969488|ref|YP_{—}001334597.1|(152969488); formate acetyltransferase 1 (Salmonella enterica subsp. enterica serovar Typhi Ty2) gi|29142384|ref|NP_{—}805726.1|(29142384) formate acetyltransferase 1 (Shigella flexneri 2a str. 301) gi|24112311|ref|NP_{—}706821.1|(24112311); formate acetyltransferase 1 (Escherichia coli O157:H7 EDL933) gi|15800764|ref|NP_{—}286778.1|(15800764); formate acetyltransferase 1 (Klebsiella pneumoniae subsp. pneumoniae MGH 78578) gi|150954337|gb|ABR76367.1|(150954337); formate acetyltransferase 1 (Yersinia pestis CA88-4125) gi|149366640|ref|ZP_{—}01888674.1|(149366640); formate acetyltransferase 1 (Yersinia pestis CA88-4125) gi|149291014|gb|EDM41089.1| (149291014); formate acetyltransferase 1 (Yersinia enterocolitica subsp. enterocolitica 8081) gi|122088805|emb|CAL11611.1|(122088805); formate acetyltransferase 1 (Shigella sonnei Ss046) gi|73854936|gb|AAZ87643.1|(73854936); formate acetyltransferase 1 (Escherichia coli UTI89) gi|91071578|gb|ABE06459.1| (91071578); formate acetyltransferase 1 (Salmonella enterica subsp. enterica serovar Typhi Ty2) gi|29138014|gb|AAO69575.1|(29138014); formate acetyltransferase 1 (Shigella boydii Sb227) gi|81246052|gb|ABB66760.1|(81246052); formate acetyltransferase 1 (Shigella flexneri 2a str. 301) gi|24051169|g|IAAN42528.1|(24051169); formate acetyltransferase 1 (Escherichia coli O157:H7 str. Sakai) gi|13360445|dbj|BAB34409.1|(13360445); formate acetyltransferase 1 (Escherichia coli O157:H7 str. Sakai) gi|15830240|ref|NP_{—}309013.1|(15830240); formate acetyltransferase I (pyruvate formate-lyase 1) (Photorhabdus luminescens subsp. laumondii TTO1) gi|36784986|emb|CAE13906.1|(36784986); formate acetyltransferase I (pyruvate formate-lyase 1) (Photorhabdus luminescens subsp. laumondii TTO1) gi|37525558|ref|NP_{—}928902.1|(37525558); formate acetyltransferase (Staphylococcus aureus subsp. aureus Mu50) gi|14245993|dbj|BAB56388.1|(14245993); formate acetyltransferase (Staphylococcus aureus subsp. aureus Mu50) gi|15923216|ref|NP_{—}370750.1|(15923216); Formate acetyltransferase (Pyruvate formate-lyase) gi|81706366|sp|Q7A7X6.1|PFLB_STAAN(81706366); Formate acetyltransferase (Pyruvate formate-lyase) gi|81782287|sp|Q99WZ7.1|PFLB_STAAM(81782287); Formate acetyltransferase (Pyruvate formate-lyase) gi|81704726|sp|Q7A1W9.1|PFLB_STAAW(81704726); formate acetyltransferase (Staphylococcus aureus subsp. aureus Mu3) gi|156720691|dbj|BAF77108.1|(156720691); formate acetyltransferase (Erwinia carotovora subsp. atroseptica SCRI1043) gi|50121521|ref|YP_{—}050688.1|(50121521); formate acetyltransferase (Erwinia carotovora subsp. atroseptica SCRI1043) gi|49612047|emb|CAG75496.1|(49612047); formate acetyltransferase (Staphylococcus aureus subsp. aureus str. Newman) gi|150373174|dbj|BAF66434.1|(150373174); formate acetyltransferase (Shewanella oneidensis MR-1) gi|24374439|ref|NP_{—}718482.1|(24374439); formate acetyltransferase (Shewanella oneidensis MR-1) gi|24349015|g|IAAN55926.1|AE015730_{—}3(24349015); formate acetyltransferase (Actinobacillus pleuropneumoniae serovar 3 str. JL03) gi|165976461|ref|YP_{—}001652054.1|(165976461); formate acetyltransferase (Actinobacillus pleuropneumoniae serovar 3 str. JL03) gi|165876562|gb|ABY69610.1|(165876562); formate acetyltransferase (Staphylococcus aureus subsp. aureus MW2) gi|21203365|dbj|BAB94066.1|(21203365); formate acetyltransferase (Staphylococcus aureus subsp. aureus N315) gi|13700141|dbj|BAB41440.1|(13700141); formate acetyltransferase (Staphylococcus aureus subsp. aureus str. Newman) gi|151220374|ref|YP_{—}001331197.1|(151220374); formate acetyltransferase (Staphylococcus aureus subsp. aureus Mu3) gi|156978556|ref|YP_{—}001440815.1|(156978556); formate acetyltransferase (Synechococcus sp. JA-2-3B′a(2-13)) gi|86607744|ref|YP_{—}476506.1|(86607744); formate acetyltransferase (Synechococcus sp. JA-3-3Ab) gi|86605195|ref|YP_{—}473958.1|(86605195); formate acetyltransferase (Streptococcus pneumoniae D39) gi|116517188|ref|YP_{—}815928.1|(116517188); formate acetyltransferase (Synechococcus sp. JA-2-3B′a(2-13)) gi|86556286|gb|ABD01243.1|(86556286); formate acetyltransferase (Synechococcus sp. JA-3-3Ab) gi|86553737|gb|ABC98695.1|(86553737); formate acetyltransferase (Clostridium novyi NT) gi|118134908|gb|ABK61952.1|(118134908); formate acetyltransferase (Staphylococcus aureus subsp. aureus MRSA252) gi|49482458|ref|YP_{—}039682.1|(49482458); and formate acetyltransferase (Staphylococcus aureus subsp. aureus MRSA252) gi|49240587|emb|CAG39244.1|(49240587), each sequence associated with the accession number is incorporated herein by reference in its entirety.

FNR transcriptional dual regulators are transcription regulators responsive to oxygen content. FNR is an anaerobic regulator that represses the expression of PDHc. Accordingly, reducing FNR will result in an increase in PDHc expression. FNR homologs and variants are known. For examples, such homologs and variants include, for example, DNA-binding transcriptional dual regulator, global regulator of anaerobic growth (Escherichia coli W3110) gi|1742191|dbj|BAA14927.1|(1742191); DNA-binding transcriptional dual regulator, global regulator of anaerobic growth (Escherichia coli K12) gi|16129295|ref|NP_{—}415850.1|(16129295); DNA-binding transcriptional dual regulator, global regulator of anaerobic growth (Escherichia coli K12) gi|1787595|gb|AAC74416.1|(1787595); DNA-binding transcriptional dual regulator, global regulator of anaerobic growth (Escherichia coli W3110) gi|89108182|ref|AP 001962.1|(89108182); fumarate/nitrate reduction transcriptional regulator (Escherichia coli UTI89) gi|162138444|ref|YP_{—}540614.2|(162138444); fumarate/nitrate reduction transcriptional regulator (Escherichia coli CFT073) gi|161486234|ref|NP_{—}753709.2|(161486234); fumarate/nitrate reduction transcriptional regulator (Escherichia coli O157:H7 EDL933) gi|15801834|ref|NP_{—}287852.1|(15801834); fumarate/nitrate reduction transcriptional regulator (Escherichia coli APEC O1) gi|117623587|ref|YP_{—}852500.1|(117623587); fumarate and nitrate reduction regulatory protein gi|71159334|sp|P0A9E5.1|FNR_ECOLI(71159334); transcriptional regulation of aerobic, anaerobic respiration, osmotic balance (Escherichia coli O157:H7 EDL933) gi|12515424|gb|AAG56466.11AE005372 1|(12515424); Fumarate and nitrate reduction regulatory protein gi|71159333|sp|P0A9E6.1|FNR_ECOL6(71159333); Fumarate and nitrate reduction Regulatory protein (Escherichia coli CFT073) gi|26108071|gb|AAN80271.1|AE016760_{—}130(26108071); fumarate and nitrate reduction regulatory protein (Escherichia coli UTI89) gi|91072202|gb|ABE07083.1|(91072202); fumarate and nitrate reduction regulatory protein (Escherichia coli HS) gi|157160845|ref|YP_{—}001458163.1|(157160845); fumarate and nitrate reduction regulatory protein (Escherichia coli E24377A) gi|157157974|ref|YP_{—}001462642.1|(157157974); fumarate and nitrate reduction regulatory protein (Escherichia coli E24377A) gi|157080004|gb|ABV19712.1|(157080004); fumarate and nitrate reduction regulatory protein (Escherichia coli HS) gi|157066525|gb|ABV05780.1|(157066525); fumarate and nitrate reduction regulatory protein (Escherichia coli APEC O1) gi|115512711|gb|ABJ00786.1|(115512711); transcription regulator Fnr (Escherichia coli O157:H7 str. Sakai) gi|13361380|dbj|BAB35338.1|(13361380) DNA-binding transcriptional dual regulator (Escherichia coli K12) gi|16131236|ref|NP_{—}417816.1|(16131236), to name a few, each sequence associated with the accession number is incorporated herein by reference in its entirety.

An acetoacetyl-coA thiolase (also sometimes referred to as an acetyl-coA acetyltransferase) catalyzes the production of acetoacetyl-coA from two molecules of acetyl-coA. Depending upon the organism used a heterologous acetoacetyl-coA thiolase (acetyl-coA acetyltransferase) can be engineered for expression in the organism. Alternatively a native acetoacetyl-coA thiolase (acetyl-coA acetyltransferase) can be overexpressed. Acetoacetyl-coA thiolase is encoded in E. coli by thl. Acetyl-coA acetyltransferase is encoded in C. acetobutylicum by atoB. THL and AtoB homologs and variants are known. For examples, such homologs and variants include, for example, acetyl-coa acetyltransferase (thiolase) (Streptomyces coelicolor A3(2)) gi|21224359|ref|NP_{—}630138.1|(21224359); acetyl-coa acetyltransferase (thiolase) (Streptomyces coelicolor A3(2)) gi|3169041|emb|CAA19239.1|(3169041); Acetyl CoA acetyltransferase (thiolase) (Alcanivorax borkumensis SK2) gi|110834428|ref|YP_{—}693287.1|(110834428); Acetyl CoA acetyltransferase (thiolase) (Alcanivorax borkumensis SK2) gi|110647539|emb|CAL17015.1|(110647539); acetyl CoA acetyltransferase (thiolase) (Saccharopolyspora erythraea NRRL 2338) gi|133915420|emb|CAM05533.1|(133915420); acetyl-coa acetyltransferase (thiolase) (Saccharopolyspora erythraea NRRL 2338) gi|134098403|ref|YP_{—}001104064.1|(134098403); acetyl-coa acetyltransferase (thiolase) (Saccharopolyspora erythraea NRRL 2338) gi|133911026|emb|CAM01139.1|(133911026); acetyl-CoA acetyltransferase (thiolase) (Clostridium botulinum A str. ATCC 3502) gi|148290632|emb|CAL84761.1|(148290632); acetyl-CoA acetyltransferase (thiolase) (Pseudomonas aeruginosa UCBPP-PA14) gi|115586808|gb|ABJ12823.1|(115586808); acetyl-CoA acetyltransferase (thiolase) (Ralstonia metallidurans CH34) gi|93358270|gb|ABF12358.1|(93358270); acetyl-CoA acetyltransferase (thiolase) (Ralstonia metallidurans CH34) gi|93357190|gb|ABF11278.1|(93357190); acetyl-CoA acetyltransferase (thiolase) (Ralstonia metallidurans CH34) gi|93356587|gb|ABE10675.1|(93356587); acetyl-CoA acetyltransferase (thiolase) (Ralstonia eutropha JMP134) gi|72121949|gb|AAZ64135.1|(72121949); acetyl-CoA acetyltransferase (thiolase) (Ralstonia eutropha JMP134)gi|72121729|gb|AAZ63915.1|(72121729); acetyl-CoA acetyltransferase (thiolase) (Ralstonia eutropha JMP134) gi|72121320|gb|AAZ63506.1|(72121320); acetyl-CoA acetyltransferase (thiolase) (Ralstonia eutropha JMP134) gi|72121001|gb|AAZ63187.1|(72121001); acetyl-CoA acetyltransferase (thiolase) (Escherichia coli) gi|2764832|emb|CAA66099.1|(2764832), each sequence associated with the accession number is incorporated herein by reference in its entirety.

Butyryl-coA dehydrogenase is an enzyme in the protein pathway that catalyzes the reduction of crotonyl-CoA to butyryl-CoA. A butyryl-CoA dehydrogenase complex (Bcd/EtfAB) couples the reduction of crotonyl-CoA to butyryl-CoA with the reduction of ferredoxin. Depending upon the organism used a heterologous butyryl-CoA dehydrogenase can be engineered for expression in the organism. Alternatively, a native butyryl-CoA dehydrogenase can be overexpressed. Butyryl-coA dehydrogenase is encoded in C. acetobuylicum and M. elsdenii by bcd. BCD homologs and variants are known. For examples, such homologs and variants include, for example, butyryl-CoA dehydrogenase (Clostridium acetobutylicum ATCC 824) gi|15895968|ref|NP_{—}349317.1|(15895968); Butyryl-CoA dehydrogenase (Clostridium acetobutylicum ATCC 824) gi|15025744|gb|AAK80657.11AE007768_{—}1|(15025744); butyryl-CoA dehydrogenase (Clostridium botulinum A str. ATCC 3502) gi|148381147|ref|YP_{—}001255688.1|(148381147); butyryl-CoA dehydrogenase (Clostridium botulinum A str. ATCC 3502) gi|148290631|emb|CAL84760.1|(148290631), each sequence associated with the accession number is incorporated herein by reference in its entirety. BCD can be expressed in combination with a flavoprotien electron transfer protein. Useful flavoprotein electron transfer protein subunits are expressed in C. acetobutylicum and M. elsdenii by a gene etfA and etfB (or the operon etfAB). ETFA, B, and AB homologs and variants are known. For examples, such homologs and variants include, for example, putative a-subunit of electron-transfer flavoprotein gi|1055221|gb|AAA95970.1|(1055221); putative b-subunit of electron-transfer flavoprotein gi|1055220|gb|AAA95969.1|(1055220), each sequence associated with the accession number is incorporated herein by reference in its entirety.

Crotonyl-coA reductase catalyzes the reduction of crotonyl-CoA to butyryl-CoA. Depending upon the organism used a heterologous Crotonyl-coA reductase can be engineered for expression in the organism. Alternatively, a native Crotonyl-coA reductase can be overexpressed. Crotonyl-coA reductase is encoded in S. coelicolor by ccr. CCR homologs and variants are known. For examples, such homologs and variants include, for example, crotonyl CoA reductase (Streptomyces coelicolor A3(2)) gi|21224777|ref|NP_{—}630556.11 (21224777); crotonyl CoA reductase (Streptomyces coelicolor A3(2)) gi|4154068|emb|CAA22721.1|(4154068); crotonyl-CoA reductase (Methylobacterium sp. 4-46) gi|168192678|gb|ACA14625.1|(168192678); crotonyl-CoA reductase (Dinoroseobacter shibae DFL 12) gi|159045393|ref|YP_{—}001534187.1| (159045393); crotonyl-CoA reductase (Salinispora arenicola CNS-205) gi|159039522|ref|YP_{—}001538775.1|(159039522); crotonyl-CoA reductase (Methylobacterium extorquens PA1) gi|163849740|ref|YP_{—}001637783.1| (163849740); crotonyl-CoA reductase (Methylobacterium extorquens PA1) gi|163661345|gb|ABY28712.1| (163661345); crotonyl-CoA reductase (Burkholderia ambifaria AMMD) gi|115360962|ref|YP_{—}778099.1| (115360962); crotonyl-CoA reductase (Parvibaculum lavamentivorans DS-1) gi|154252073|ref|YP_{—}001412897.1| (154252073); Crotonyl-CoA reductase (Silicibacter sp. TM1040) gi|99078082|ref|YP_{—}611340.1| (99078082); crotonyl-CoA reductase (Xanthobacter autotrophicus Py2) gi|154245143|ref|YP_{—}001416101.1| (154245143); crotonyl-CoA reductase (Nocardioides sp. JS614) gi|119716029|ref|YP_{—}922994.1|(119716029); crotonyl-CoA reductase (Nocardioides sp. JS614) gi|119536690|gb|ABL81307.1|(119536690); crotonyl-CoA reductase (Salinispora arenicola CNS-205) gi|157918357|gb|ABV99784.1| (157918357); crotonyl-CoA reductase (Dinoroseobacter shibae DFL 12) gi|157913153|gb|ABV94586.1| (157913153); crotonyl-CoA reductase (Burkholderia ambifaria AMMD) gi|115286290|gb|ABI91765.1| (115286290); crotonyl-CoA reductase (Xanthobacter autotrophicus Py2) gi|154159228|gb|ABS66444.1| (154159228); crotonyl-CoA reductase (Parvibaculum lavamentivorans DS-1) gi|154156023|gb|ABS63240.1| (154156023); crotonyl-CoA reductase (Methylobacterium radiotolerans JCM 2831) gi|170654059|gb|ACB23114.1|(170654059); crotonyl-CoA reductase (Burkholderia graminis C4D1M) gi|170140183|gb|EDT08361.1| (170140183); crotonyl-CoA reductase (Methylobacterium sp. 4-46) gi|168198006|gb|ACA19953.1|(168198006); crotonyl-CoA reductase (Frankia sp. EAN1pec) gi|158315836|ref|YP_{—}001508344.1|(158315836), each sequence associated with the accession number is incorporated herein by reference in its entirety.

In yet other embodiment, in addition to any of the foregoing and combinations of the foregoing, additional genes/enzymes may be used to produce a desired product. For example, the following table provide enzymes that can be combined with the NOG pathway enzymes for the production of 1-butanol:

| | Exemplary | | Exemplary |
|---|---|---|---|
| Enzyme | Gene(s) | 1-butanol | Organism |
| Ethanol Dehydrogenase | adhE | − | E. coli |
| Lactate Dehydrogenase | ldhA | − | E. coli |
| Fumarate reductase | frdB, frdC, | − | E. coli |
| | or frdBC | | |
| Oxygen transcription | fnr | − | E. coli |
| regulator | | | |
| Phosphate | pta | − | E. coli |
| acetyltransferase | | | |
| Formate | pflB | − | E. coli |
| acetyltransferase | | | |
| acetyl-coA | atoB | + | C. acetobutylicum |
| acetyltransferase | | | |
| acetoacetyl-coA | thl, thlA, | + | E. coli, |
| thiolase | thlB | | C. acetobutylicum |
| 3-hydroxybutyryl-CoA | hbd | + | C. acetobutylicum |
| dehydrogenase | | | |
| crotonase | crt | + | C. acetobutylicum |
| butyryl-CoA | bcd | + | C. acetobutylicum, |
| dehydrogenase | | | M. elsdenii |
| electron transfer | etfAB | + | C. acetobutylicum, |
| flavoprotein | | | M. elsdenii |
| aldehyde/alcohol | adhE2 | + | C. acetobutylicum |
| dehydrogenase (butyral- | bdhA/bdhB | | |
| dehyde | aad | | |
| dehydrogenase/butanol | | | |
| dehydrogenase) | | | |
| crotonyl-coA reductase | ccr | + | S. coelicolor |
| trans-2-enoyl-CoA | Ter | + | T. denticola, |
| reductase | | | F. succinogenes |

* knockout or a reduction in expression are optional in the synthesis of the product, however, such knockouts increase various substrate intermediates and improve yield.

In addition, and as mentioned above, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

In some instances “isozymes” can be used that carry out the same functional conversion/reaction, but which are so dissimilar in structure that they are typically determined to not be “homologous”. For example, glpX is an isozyme of fbp, tktB is an isozyme of tktA, talA is an isozyme of talB and rpiB is an isozyme of rpiA.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Culture conditions suitable for the growth and maintenance of a recombinant microorganism provided herein are described in the Examples below. The skilled artisan will recognize that such conditions can be modified to accommodate the requirements of each microorganism. Appropriate culture conditions useful in producing a acetyl-phosphate, acetyl-CoA or other metabolites derived therefrom including, but not limited to 1-butanol, n-hexanol, 2-pentanone and/or octanol products comprise conditions of culture medium pH, ionic strength, nutritive content, etc.; temperature; oxygen/CO₂/nitrogen content; humidity; light and other culture conditions that permit production of the compound by the host microorganism, i.e., by the metabolic action of the microorganism. Appropriate culture conditions are well known for microorganisms that can serve as host cells.

It is understood that a range of microorganisms can be modified to include a recombinant metabolic pathway suitable for the production of n-butanol, n-hexanol and octanol. It is also understood that various microorganisms can act as “sources” for genetic material encoding target enzymes suitable for use in a recombinant microorganism provided herein.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ([NaCl]); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The disclosure includes recombinant microorganisms that comprise at least one recombinant enzymes of the NOG pathway set forth in FIG. 1. For example, chemoautotrophs, photoautotroph, and cyanobacteria can comprise native F/Xpk enzymes, accordingly, overexpressing FPK, XPK, or F/Xpk by tying expression to a non-native promoter can produce sufficient metabolite to drive the NOG pathway. Additional enzymes can be recombinantly engineered to further optimize the metabolic flux, including, for example, balancing ATP, NADH, NADPH and other cofactor utilization and production.

In one embodiment, E. coli can be engineered with the NOG pathway and further engineered to produce acetate. For example, because E. coli does not have an endogenous F/Xpk, one may express a phosphoketolase such as the one from Bifidobacterium adolescentis. Additionally, fructose-6-phosphate bisphosphatase (an endogenous gluconeogeneic enzyme) needs to be active during NOG thus expression of a FBPase in sugar-containing medium could be beneficial. There are two classes of FBPases in E. coli which are known as fbp and glpX. They are completely different in sequence and structure, yet they share a similar function (e.g., as isozymes). It is possible that instead of expressing these enzymes, one can change the regulation/inhibition so that FBPase is active in NOG. The reverse reaction of FBPase, phosphofructokinase (pfkA or pfkB), can serve as a driving force to consume excess ATP produced, if acetate is the final product. Otherwise, pfkAB can be removed to minimize ATP consumption. If glucose is the initial carbon source, one may avoid the PTS glucose transport system which requires phosphoenolpyruvate (PEP). Since NOG does not produce PEP (unlike regular EMP glycolysis), an alternative ATP-dependent transport system may be used. For example, one could use the ABC-type galactose permase transporter which can also actively transport glucose into the E. coli cell. Then to phosphorylate glucose, glucokinase (glk) can be expressed. To minimize flux through EMP glycolysis, one could knockout glyceraldehyde-3-phosphate dehydrogenase (gapA) which is typically considered an essential gene. Additionally, to maximize flux through NOG one may knockout undesired competing reaction such as lactate dehydrogenase (ldhA), fumarate reductase (frdABCD). If acetate is the desired product, one could remove alcohol dehydrogenase (adhE). If acetate is not the final product, one could remove acetate kinase (ackA). Thus to convert glucose to 3 acetate in E. coli, one could (a) express a Phosphoketolase and a fructose-6-phosphate bisphosphatase f/xpk and fbp (and/or glpX); express an ATP-dependent glucose transport system galP+glk; and optionally remove competing pathways such as ptsG, gapA, ldhA, adhE, frdABCD.

In yeast, such as S. cerevisiae, glucose is phosphorylated by glucokinase instead of the PTS transporter. This avoids the need to use galP and glk. S. cerevisiae has an FBPase which is quickly degraded by catabolite repression under glucose conditions. Thus, removing this degradation and overexpressing a FBP would be beneficial for NOG to work. Since S. cerevisiae does not have an endogenous F/Xpk, one may express a phosphoketolase such as the one from Bifidobacterium adolescentis. Furthermore, since S. cerevisiae naturally produces ethanol, from pyruvate, rather than acetyl-coA, one would need to convert acetyl-phosphate to acetyl-coA, which is reduced to acetaldehyde and then ethanol. Thus, the enzymes PTA and AdhE need to be expressed in the cytosol of yeast to accomplish these reactions. The native pyruvate carboxylase may be removed to minimize CO₂ in ethanol production. To minimize flux through traditional glycolysis pathway, one could knockout glyceraldehyde-3-phosphate dehydrogenase. Other competing pathway can be removed such as glycerol dehydrogenase, and acetyl-coA synthetase. To produce 3 moles of ethanol from glucose, additional reducing equivalents (such as from NADH) need to be supplied by using an external electron donor, such as hydrogen, CO, or formate. The theoretical conversion would require an additional six reduced equivalents from glucose to three ethanol. In the case of hydrogen, a hydrogenase may be expressed to convert hydrogen to NADH. In the case of CO, a carbon monoxide dehydrogenase may be expressed to generate NADH. If formate is used, a formate dehydrogenase may be expressed to convert formate to NADH and CO₂. Thus, in one embodiment, to make 3 ethanol from glucose in S. cerevisiae one would supply formate and express formate dehydrogenase to supply NADH; express an f/xpk and FBPase; remove glycerol dehydrogenase, acetyl-coa synthetase, glyceraldehyde-3-phosphate dehydrogenase, and pyruvate decarboxylase; and express pta and AdhE.

In another embodiment, a method of producing a recombinant microorganism that comprises optimized carbon utilization including a non-oxidative sugar utilization that converts a suitable carbon substrate to acetyl-phosphate, acetyl-CoA or other metabolites derived therefrom including, but not limited to, 1-butanol, 2-pentanone, isobutanol, n-hexanol and/or octanol is provided. The method includes transforming a microorganism with one or more recombinant polynucleotides encoding polypeptides selected from the group consisting of a fructose-6-phosphate phosphoketolase activity, a xylulose-5-phosphate phosphoketolase activity, a transaldolase activity, a transketolase activity, a ribose-5-phosphate isomerase activity, ribulose-5-phosphate epimerase activity, a triose phosphate isomerase activity, a fructose 1,6-bisphosphate aldolase activity, a fructose 1,6-bisphosphatase activity, a keto thiolase or acetyl-CoA acetyltransferase activity, hydroxybutyryl CoA dehydrogenase activity, crotonase activity, crotonyl-CoA reductase or butyryl-CoA dehydrogenase activity, trans-enoyl-CoA reductase and alcohol dehydrogenase activity.

In another embodiment, as mentioned previously, a recombinant organism as set forth in any of the embodiments above, is cultured under conditions to express any/all of the enzymatic polypeptide and the culture is then lysed or a cell free preparation is prepared having the necessary enzymatic activity to carry out the pathway set forth in FIG. 1 and/or the production of a 1-butanol, isobutanol, n-hexanol, octanol, 2-pentanone among other products.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13:563-564.

Improved methods for cloning in vitro amplified nucleic acids are described in Wallace et al., U.S. Pat. No. 5,426,039.

Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The invention is illustrated in the following examples, which are provided by way of illustration and are not intended to be limiting.

### Examples

To construct an in vitro system, all the NOG enzymes were acquired commercially or purified by affinity chromatography (FIG. 10), tested for activity (FIG. 11), and mixed together in a properly selected reaction buffer. The system was ATP- and redox-independent and comprised eight enzymes: Fpk/Xpk, Fbp, fructose bisphosphate aldolase (Fba), triose phosphate isomerase (Tpi), ribulose-5-phosphate 3-epimerase (Rpe), ribose-5-phosphate isomerase (Rpi), transketolase (Tkt), and transaldolase (Tal). AcP concentration was measured using an end-point colorimetric hydroxamate method. Using this in vitro system an initial 10 mM F6P was completely converted to stoichiometric amounts of AcP (within error) at room temperature after 1.5 hours (FIG. 4C). As a control, when no Tal was added, only one-third of the AcP was produced (FIG. 4C).

To extend the production further to acetate, Ack was be added to the in vitro NOG system. On the basis of the simulation discussed above, phosphofructokinase was also added to maintain ATP-balance. Since the ADP (the substrate for acetate kinase) is regenerated, only a catalytic amount (20 μM) was necessary. Acetate concentration monitored by HPLC showed maximum conversion (FIG. 4D), which was three-times higher than that produced by the control with no Tal added. Without the complete NOG, F6P was converted to equilimolar amounts of E4P and acetate in a linear pathway. Since the core portion of NOG can convert any sugar phosphate (triose to sedoheptulose) to stoichiometric amounts of AcP, similar in vitro systems were tested on ribose-5-phosphate and G3P. These two compounds produced nearly theoretical amounts of acetyl-phosphate at 2.3 and 1.6 mM of AcP per mM of substrate, respectively (FIG. 4E).

After demonstrating the feasibility of NOG using in vitro enzymatic systems, NOG was engineered into Escherichia coli. Xylose was used because it avoids the complication of various glucose-mediated regulations, including the use of phosphotransferase system for transport. In order to engineer NOG for xylose in E. coli, two enzymes were overexpressed: F/Xpk (encoded by f/xpk from Bifidobacterium adolescentis) and Fbp (encoded by E. coli fbp). Other enzymes in NOG were natively expressed in E. coli under the experimental conditions. The genes encoding these two enzymes were cloned on a high copy plasmid (pIB4) under the control of the PLlacO-1 IPTG-inducible promoter (FIG. 5A). The plasmid was transformed into three E. coli strains: JCL16 [wild type], JCL166 [ΔldhA, ΔadhE, Δfrd], and JCL 118 [ΔldhA, ΔadhE, Δfrd, ΔpflB]. The latter two strains were used to avoid pathways competing with the synthetic NOG (FIG. 5B). The expression of F/Xpk and Fbp was demonstrated by protein electrophoresis (FIG. 12) and their activities were confirmed by a coupled enzyme assay (FIG. 5C). After an initial aerobic growth phase for cell growth, high cell density cells were harvested and re-suspended in anaerobic minimal medium with xylose at a final OD₆₀₀ of 9. Anaerobic conditions were used to avoid the oxidation of acetate through the TCA cycle. HPLC was used for monitoring xylose consumption and organic acids formation. The wild-type host (JCL16) produced a mixture of lactate, formate, succinate, and acetate from xylose, and the yield on acetate was quite low at about 0.4 acetates produced per xylose consumed, indicating that EMP and other fermentative pathways out-competed the synthetic NOG. By removing several fermentative pathways by the Δldh, ΔadhE, and Δfrd knockouts in JCL166, the yield was increased to 1.1 acetate/xylose consumed. After further deleting pflB in JCL118, the yield reach the highest level of 2.2 acetates/xylose consumed, approaching the theoretical maximum of 2.5 mole of acetate/mole of xylose (FIG. 5D). Some succinate remained, presumably due to succinate dehydrogenase left over from the aerobic growth phase. Note that without NOG, the theoretical maximum of acetate production from xylose is 2.5 mole of acetate/mole of xylose, indicating that the synthetic NOG in this strain effectively outcompeted native pathways.

One important enzyme in NOG is the irreversible Fpk/Xpk which can split F6P or xylulose-5-phosphate into AcP and E4P or G3P, respectively. This class of enzymes has been well-characterized in heterofermentative pathways from Lactobacillae and Bifidobacteria. In Lactobacillae, glucose is first oxidized and decarboxylated to form CO₂, reducing power, and xylulose-5-phosphate, which is later split to AcP and G3P. Xpks have also been found in Clostridium acetobutylicum where up to 40% of xylose is degraded by the phosphoketolase pathway. Bifidobacteria, utilizes the Bifid Shunt, which oxidizes two glucoses into two lactates and three acetates. This process yields increase the ATP yield to 2.5 ATP/glucose. In both variants G3P continues through the oxidative EMP pathway to form pyruvate (FIG. 13). Thus these pathways are still oxidative and are not able to directly convert glucose to three two-carbon compounds. For NOG to function, Fpk/Xpk and Fbp must be simultaneously expressed. However, since Fbp is a gluconeogenic enzyme, it is typically not active in the presence of glucose. Thus, although these organisms have all the genes necessary for NOG, it is unlikely that NOG is functional in these organisms in the presence of glucose.

Since the CBB cycle contains all the enzymes besides Fpk/Xpk necessary for NOG, it is likely that these organisms can be readily engineered to make acetyl-CoA by combining NOG with CBB (FIG. 3A). This would result in a 50% increase in carbon fixation efficiency to produce one acetyl-CoA compared with the traditional oxidative pyruvate route. In view of the relatively low turnover number of Rubisco, increased output per CO₂ fixation event would be beneficial.

The NOG pathway described above can take any sugar as input molecules, as long as it can be converted to sugar phosphates that are present in the carbon rearrangement network. FIGS. 6a and 6b show the pathways using pentose or triose sugar phosphates as inputs. These pathways use F/Xpk. Similar pathways can be drawn using Fpk only or Xpk only. Enzyme abbreviations and EC numbers are listed in Table A.

TABLE A
Enzyme abbreviations and EC numbers:

| Name | | Abbrev. | EC# | Verified Source |
|---|---|---|---|---|
| F6P-Phosphoketolase | 1a | Fpk | 4.1.2.22 | B. adolescentis* |
| X5P-Phosphoketolase | 1b | Xpk | 4.1.2.9 | L. plantarum |
| Transaldolase | 2 | Tal | 2.2.1.2 | E. Coli |
| Transketolase | 3 | Tkt | 2.2.1.1 | E. Coli |
| Triose Phosphate Isomerase | 6 | Tpi | 5.3.1.1 | E. Coli |
| Fructose 1,6 Bisphosphatase | 8 | Fbp | 3.1.3.11 | E. Coli |
| Fructose 1,6 bisphosphate | 7 | Fba | 4.1.2.13 | E. Coli |
| Aldolase | | | | |
| Ribose-5-phosphate isomerase | 4 | Rpi | 5.3.1.6 | E. Coli |
| Ribulose-3-phosphate | 5 | Rpe | 5.1.3.1 | E. Coli |
| epimerase | | | | |
| Glucokinase | | Glk | 2.7.1.2 | E. Coli |
| Glucose-6-phosphate | | Zwf | 1.1.1.49 | E. Coli |
| Dehydrogenase | | | | |
| Phosphoglucose isomerase | | Pgi | 5.3.1.9 | E. Coli |
| Acetate Kinase | | Ack | 2.7.2.1 | E. Coli |
| Hexulose-6-phosphate synthase | | Hps | 4.1.2.43 | M. capsulatus |
| Hexulose 6-phosphate | | Phi | 5.3.1.27 | M. Capsulatus |
| isomerase | | | | |
| Dihydroxyacetone synthase | | Das | 2.2.1.3 | C. boindii |
| (formaldehyde transketolase) | | | | |
| Phosphotransacetylase | | Pta | 2.3.1.8 | E. Coli |
| Methanol dehydrogenase | | Mdh | 1.1.99.37 | B. Methanolicus |

Thermodynamics of NOG Enzymes.

The change in standard Gibbs free energy (ΔrG′° in kJ/mol) for each step was calculated using eQuilibrator with pH=7.5 and ionic strength=0.2 M to represent E. coli's cytosolic environment (FIG. 7). All values were obtained using the difference of the standard Gibbs free energy of formation between the products and reactants. Since standard state is set at 1 M for all reactants (including water), some of the values do not correspond with experimentally verified data. For example the calculations show that Fba has a larger free energy drop than Fbp, even though Fba is known to be reversible and Fbp is irreversible. When using 1 mM for all reactants, the adjusted ΔrG′ for both fructose-1,6-bisphosphatase and fructose-1,6-bisphosphate aldolase change dramatically and closer represent reality. Nevertheless, the calculation at standard free energy gives some useful insight into the overall thermodynamics of NOG and EMP.

Combination of NOG with the Dihydroxyacetone (DHA) Pathway.

NOG can be combined with the DHA pathway, which is analogous to the RuMP pathway for assimilation of formaldehyde. The pathways are shown in FIGS. 8a and 8b. This pathway depends on the action of the gene fructose-6-phosphate aldolase (fsa) which has been characterized from E. coli. Though the native activity of this enzyme was reported to have a high Kₘ, recent design approaches have improved affinity towards DHA. The overall pathway from two methanol to ethanol is favorable with a ΔrG′°=−68.2 kJ/mol.

Kinetic Simulations of Non-Oxidative Glycolysis.

A kinetic models was used to test the feasibility and robustness of NOG. Ordinary differential equations were constructed to simulate the dynamics of NOG in vitro. The open-source program COPASI was used to simulate the system. For example, the simulation result shown in FIG. 4a was generated by simulating the reaction network in FIG. 9a, which is the core NOG reactions shown in FIG. 1a. To investigate the effect of the phosphoketolase activity on final product accumulation, a batch approach was used where a certain amount of initial substrate (F6P) is allowed to react for a long time and final concentrations are measured. This represents the in vitro assay where a specific amount of substrate is added to purified enzymes. Since the overall pathway involves many enzymes and some of their detailed mechanisms remain unknown, Michaelis-Menten kinetics for irreversible enzymes (phosphoketolases and Fbp) and mass action kinetics for all reversible steps (Tkt, Tal, Rpe, Rpi, Fba, Tpi) was assumed (FIG. 9b). For irreversible steps, the Kₘ was set to 0.1 mM and Vₘₐₓ to 0.01 mM/sec. The reversible reactions had a forward and reverse rate of 1/sec. Changing the kinetic parameters for any reaction except for phosphoketolase did not affect the pathway performance. The ordinary differential equations (ODEs) for the system are shown in FIG. 9c, as an illustration. By running a parameter scan on phosphoketolase activity, the final concentration of AcP and E4P/G3P was plotted as a function of Vₘₐₓ for Fpk or Xpk activity (from 0.0001 to 1 mM/sec). As expected, the stoichiometric conversion of F6P to three AcP can be achieved using either only Fpk or only Xpk, however high Fpk activity posed a problem of intermediate accumulation. Once Fpk activity is too high, it out competes the rest of the NOG pathway which causes the F6P to degrade too quickly leaving E4P to be stuck. When dual Fpk and Xpk is modeled, the accumulation of E4P does not occur as long as Xpk activity is at least 10 times greater than Fpk activity.

To simulate one of the applications of NOG, the conversion of xylose to 2.5 acetate was modeled by adding four more enzymes (XylA, XylB, Ack, and Pfk). XylA corresponds to xylose isomerase and XylB is xylulokinase which are involved in the conversion of xylose to xylulose-5-phosphate. Phosphofructokinase (Pfk) was added to create an ATP futile cycle consisting of Fbp and Pfk. Together these two enzymes act as an ATPase which is necessary to maintain ATP balance since the production of acetate from xylose produces a net of 1.5 ATP. If ATP was not returned back to ADP, then Ack would not be able to catalyze the reaction of AcP to Acetate. A parameter scan of Pfk activity showed that very low or very high ATP degradation is detrimental to pathway performance. This is because xylulokinase requires some amount ATP, while acetate kinase requires ADP for the forward direction.

Obtaining and Purifying all NOG Enzymes.

Six proteins (Fba, Glk, Zwf, Tpi, Pgi, and Pfk) were purchased from Sigma-Aldrich while the rest (Tkt, Tal, Rpe, Rpi, Ack, Fbp, and F/Xpk) were purified in-house since they were not commercially available in reasonable quantities. All commercial enzymes were purchased from Sigma Chemical Co. (St. Louis, Mo.). Rabbit muscle was the source for Tpi and Fba, Baker's yeast for Glk, Zwf, and Pgi, and Bacillus stearothermophilus for Pfk.

All non-commercial proteins were put on the high expression plasmid pQE9 (Qiagen, Chatsworth, Calif.) with an N-terminal 6× histidine tag and cloned into XL1-Blue (Stratagene). Expression in the same cloning strain yielded high yields when cells were induced at an OD of 0.4-0.6 and induced at 0.1 mM IPTG for four hours. The purification was done according to the protocol listed in His-Spin Protein Miniprep kit (Zymo Research, Orange, Calif.). All of the genetic sequences except F/Xpk were taken from E. coli's JCL16 gDNA. Specifically, rpe, rpiA, tktA, talB, ackA, and fbp were cloned from E. coli. F/Xpk was cloned from Bifidobacterium adolenscentis (ATCC 15703 gDNA). Between 0.5-3 milligrams of protein was obtained from each elution and the purity was analyzed by SDS-PAGE by loading 10 uL of diluted protein sample using the MINI PROTEAN II (Bio-Rad Laboratories, Hercules, Calif.). FIG. 10 shows the SDS agarose gel electrophoresis of the purified proteins.

Enzyme Assays.

To verify the activity of each purified enzyme, a system of several NADPH-linked coupled assays was designed. Using the “Enzyme Buffer” consisting of 50 mM 3-(N-morpholino)propanesulfonic acid (MOPS) pH 7.5, 5 mM MgCl₂, and 1 mM TPP, using the commercial enzymes described above (Glk, Zwf, and Pgi) high activity was established. The Zwf linked assay was chosen since the production of NADPH produces less noise then the degradation of NADH by glycerol-3-phosphate dehydrogenase. All the coupled assays ended with the formation of G6P, which becomes oxidized by glucose-6 phosphate dehydrogenase (Zwf) to 6-phospho D-glucono-1,5-lactone (PGL) as shown in FIG. 11. All the assays were done in the same “Enzyme Buffer” with two controls (no enzymes and no substrate). The initial substrate concentration was chosen at 10 mM to ensure that enzymes with high Km (such as F/Xpk) would still have high activity. Assays that involved Ack, only required a catalytic amount of ADP (20 uM) since the cofactor become recycled by glucokinase (Glk). Additionally, high concentrations of ADP or ATP was found to inhibit fructose-bisphosphatase (Fbp), thus low cofactor concentration was beneficial.

In Vitro NOG for Converting F6P to AcP.

To construct an NOG system in vitro, the following enzymes were used: HIS-F/Xpk (from Bifidobacterium adolenscentis), HIS-tktA, HIS-talB, HIS-rpe, HIS-rpi, (all from E. coli), One unit (one micromole of product per minute) of Tal, Tkt, and Fba and 0.1 unit of F/Xpk was added. Excess amounts of the highly active isomerases (Rpe, Rpi, and Tpi) were added to the enzyme buffer as described above. Thiamine pyrophosphate (TPP) is a necessary cofactor for F/Xpk and Tkt (both enzymes are structurally similar). The in vitro NOG was initiated by addition of the initial substrate, F6P, to a final concentration of 10 mM, and the reaction mixture (500 uL) was incubated at room temperature. As a negative control, all the enzymes except Tal were used in the reaction. As expected, NOG could not proceed to completion without Tal.

Samples were taken every 30 min to measure acetyl-phosphate concentration, which was carried out using the hydroxamate method. At each time point, 40 uL of reaction mixture was taken out and 60 uL of hydroxamate HCl (2M pH 6.5) was added. After waiting 10 minutes at room temperature, 40 uL of TCA (15%), 40 uL HCl (4M), and 40 uL of FeCl₃ (in 0.1 M HCl) was added. The absorbance was measured at 420 nm and concentration was fit to an acetyl-phosphate standard.

In Vitro NOG for Converting F6P to Acetate.

To extend the production from F6P to acetate, the same buffer with two more enzymes (Ack purified in-house) and Pfk (commercial enzyme) was used. A catalytic amount of ATP was added at 0.02 mM since ADP becomes regenerated from Pfk. The reaction mixture was incubated at room temperature for three hours after which the sample was analyzed by HPLC. The organic acid column Aminex HPX-87H was used with 5 mM H₂SO₄ as the running buffer at 35° C. and 0.6 mL/min flow rate.

Construction of In Vivo NOG.

For the in vivo production of acetate from xylose, the plasmid pIB4 was made using pZE12 as the vector, F/Xpk from B. adolenscentis and Fbp from E. coli (JCL16 gDNA). The strains JCL16, JCL166, and JCL118 were constructed (see, e.g., Int'l Patent Publication No. WO 2012/099934). This was done using the P1 phage transduction method with the Keio collection as the template for single-gene knockouts. The strains JCL166 and JCL118 were transformed with pIB4. Single colonies were grown in LB medium overnight and inoculated into fresh LB+1% xylose culture the next day. After reaching an OD=0.4-0.6, the strains were induced with 0.1 mM IPTG. After overnight induction, the cells were concentrated ten-fold and resuspended anaerobically in M9 1% xylose. A small portion of the induced cells was extracted for HIS-tag purification to verify the activity of F/Xpk and Fbp, and the rest was incubated anaerobically overnight for acetate production. The final mixture was spin down at 14,000 rpm, and a diluted supernatant was run on HPLC to measure xylose and organic acid concentration. The expression of F/Xpk and Fbp are shown in FIG. 12.

Phosphoketolase in Nature.

Phosphoketolase have been known to exist in many bacteria such as Bifidobacteria for decades. Bifidobacteria make up a large portion of the beneficial flora in human's stomach, are used in the fermentation of various foods from yogurt to kimchi, and are even sold in a dehydrated pill form. These bacteria contain a unique pathway that can ferment sugars to a mixture of lactate and acetate. By using the F6P/X5P phosphoketolase enzyme, they are able to obtain more ATP than other fermentative pathways at 2.5 ATP/glucose (See, e.g., FIG. 13).

A number of embodiments of the invention have been described. Nevertheless, it will be understood that various modifications may be made without departing from the spirit and scope of the invention. Accordingly, other embodiments are within the scope of the following claims.

Chemoautotrophs, photoautotroph, cyanobacteria overexpress FPK, XPK, tied to non-native promoter.

What is claimed is:

1. A recombinant microorganism comprising a non-CO₂ evolving metabolic pathway for the synthesis of acetyl phosphate with improved carbon yield beyond 1:2 molar ratio (fructose 6-phosphate:Acetyl phosphate) from a carbon substrate using a pathway comprising an enzyme having fructose-6-phosphoketolase (Fpk) activity and/or xylulose-5-phosphoketolase (Xpk) activity.

2. The recombinant microorganism of claim 1, wherein the microorganism can convert any sugar phosphate to acetyl phosphate with improved yield beyond those obtained by pathways that involve pyruvate decarboxylation.

3. The recombinant microorganism of claim 2, wherein the sugar phosphate is selected from the group consisting of: sugar phosphates of a triose (G3P, DHAP), an erythrose (E4P), a pentose (RSP, Ru5P, RuBP, X5P), a hexose (F6P, H6P, FBP, G6P), and a sedoheptulose (S7P, SBP).

4. The recombinant microorganism of claim 3, wherein the sugar phosphates are derived from methanol, methane, CO₂, CO, formaldehyde, formate, glycerol, a carbohydrate having the general formula CHₙH₂ₙOₙ, wherein n=3 to 7, or cellulose as a carbon source.

5. The recombinant microorganism of claim 1, wherein the microorganism is yeast.

6. The recombinant microorganism of claim 1, wherein the microorganism is a prokaryote.

7. The recombinant microorganism of claim 6, wherein the microorganism is derived from an E. coli microorganism.

8. The recombinant microorganism of claim 7, wherein the E. coli is engineered to express a phosphoketolase.

9. The recombinant microorganism of claim 1, wherein the phosphoketolase is Fpk, Xpk or a bifunctional F/Xpk enzyme.

10. The recombinant microorganism of claim 1, wherein the microorganism is engineered to heterologously expresses one or more of the following enzymes:
(a) a phosphoketolase (F/Xpk);
(b) a transaldolase (Tal);
(c) a transketolase (Tkt);
(d) a ribose-5-phosphate isomerase (Rpi);
(e) a ribulose-5-phosphate epimerase (Rpe);
(f) a triose phosphate isomerase (Tpi);
(g) a fructose 1,6 bisphosphate aldolase (Fba);
(h) a sedoheptulose bisphosphate aldolase (Sba);
(i) a fructose 1,6 bisphosphatase (Fbp); and
(j) a sedoheptulose 1,6, bisphosphatase (Sbp).

11. The recombinant microorganism of claim 9, wherein the microorganism is engineered to express a phosphoketolase derived from Bifidobaceterium adolescentis.

12. The recombinant microorganism of claim 11, wherein the phosphoketolase comprises a sequence that is at least 49% identical to SEQ ID NO:2 and has phosphoketolase activity.

13. The recombinant microorganism of claim 9, wherein the microorganism is engineered to express or over express a fructose 1,6 bisphosphatase.

14. A recombinant microorganism comprising a non-CO₂-evolving pathway that comprises synthesizing acetyl phosphate using a recombinant metabolic pathway that metabolizes methanol, methane, formate, formaldehyde, CO₂, CO, a carbohydrate having the general formula CₙH₂ₙOₙ wherein n=3 to 7, or a sugar phosphate metabolite, with improved carbon yield beyond those obtained by pathways that involve pyruvate decarboxylation.

15. The recombinant microorganism of claim 14, wherein the microorganism can convert any sugar phosphate to acetyl phosphate with improved carbon yield beyond those obtained by pathways that involve pyruvate decarboxylation.

16. The recombinant microorganism of claim 14, wherein the sugar phosphate is selected from the group consisting of: sugar phosphate of a triose (G3P, DHAP), a erythrose (E4P), a pentose (R5P, Ru5P, X5P), a hexose (F6P, H6P, FBP, G6P), and a sedoheptulose (S7P, SBP).

17. The recombinant microorganism of claim 14, wherein the microorganism is a yeast.

18. The recombinant microorganism of claim 14, wherein the microorganism is a prokaryote.

19. The recombinant microorganism of claim 18, wherein the microorganism is derived from an E. coli parental microorganism.

20. The recombinant microorganism of claim 14, wherein microorganism is engineered to express a phosphoketolase.

21. The recombinant microorganism of claim 20, wherein the phosphoketolase is Fpk, Xpk or a bifunctional F/Xpk enzyme.

22. The recombinant microorganism of any of claim 14, wherein the microorganism is engineered to heterologously expresses one or more of the following enzymes:
(a) a phosphoketolase;
(b) a transaldolase;
(c) a transketolase;
(d) a ribose-5-phosphate isomerase;
(e) a ribulose-5-phosphate epimerase;
(f) a triose phosphate isomerase;
(g) a fructose 1,6 bisphosphate aldolase;
(h) a sedoheptulose bisphosphate aldolase
(i) a fructose 1,6 bisphosphatase; and
(j) a sedoheptulose 1,6, bisphosphatase.

23. The recombinant microorganism of claim 20, wherein the microorganism is engineered to express a phosphoketolase derived from Bifidobaceterium adolescentis.

24. The recombinant microorganism of claim 23, wherein the phosphoketolase comprises a sequence that is at least 49% identical to SEQ ID NO:2 and has phosphoketolase activity.

25. The recombinant microorganism of any of claim 14, wherein the microorganism is engineered to express or over express a fructose 1,6 bisphosphatase.

26. A recombinant microorganism comprising a pathway that produces acetyl-phosphate through carbon rearrangement of E4P and metabolism of a carbon source selected from methanol, methane, formate, formaldehyde, CO₂, CO, a carbohydrate (CₙH₂ₙOₙ, n=3-7) or a sugar phosphate.

27. The recombinant microorganism of claim 26, wherein the microorganism can convert any sugar phosphate to acetyl phosphate with improved carbon yield beyond those obtained by pathways that involve pyruvate decarboxylation.

28. The recombinant microorganism of claim 26, wherein the microorganism uses methanol or methane to produce F6P which is then used as a carbon source for stoichiometric production of acetyl phosphate.

29. The recombinant microorganism of claim 26, wherein the microorganism is a prokaryote or eukaryote.

30. The recombinant microorganism of claim 29, wherein the microorganism is derived from an E. coli microorganism.

31. The recombinant microorganism of claim 30, wherein the E. coli is engineered to express a phosphoketolase.

32. The recombinant microorganism of claim 31, wherein the phosphoketolase is Fpk, Xpk or a bifunctional F/Xpk enzyme.

33. The recombinant microorganism of claim 26, wherein the microorganism is engineered to heterologously expresses one or more of the following enzymes:
(a) a phosphoketolase;
(b) a transaldolase;
(c) a transketolase;
(d) a ribose-5-phosphate isomerase;
(e) a ribulose-5-phosphate epimerase;
(f) a triose phosphate isomerase;
(g) a fructose 1,6 bisphosphate aldolase;
(h) a sedoheptulose bisphosphate aldolase
(i) a fructose 1,6 bisphosphatase; and
(j) a sedoheptulose 1,6, bisphosphatase.

34. The recombinant microorganism of claim 31, wherein the microorganism is engineered to express a phosphoketolase derived from Bifidobaceterium adolescentis.

35. The recombinant microorganism of claim 34, wherein the phosphoketolase comprises a sequence that is at least 49% identical to SEQ ID NO:2 and has phosphoketolase activity.

36. The recombinant microorganism of claim 26, wherein the microorganism is engineered to express or over express a fructose 1,6 bisphosphatase.

37. The recombinant microorganism of claim 26, wherein the carbon rearrangement comprises an enzymatic reaction by an enzymes selected from the group consisting of a transaldolase, ribulose-5-phosphate epimerase, ribulose 5-phosphate isomerase, fructose 1,6 bisphosphate aldolase, fructose 1,6 bisphosphatase, sba, sbp and any combination thereof.

38. A recombinant microorganism expressing enzymes that catalyze the conversion described in (i)-(ix), wherein at least one enzyme or the regulation of at least one enzyme that performs a conversion described in (i)-(ix) is heterologous to the microorganism:
(i) the production of acetyl-phosphate and erythrose-4-phosphate (E4P) from fructose-6-phosphate and/or the production of acetyl-phosphate and glyceraldehyde 3-phosphate (G3P) from xylulose 5-phosphate;
(ii) the conversion of fructose-6-phosphate and E4P to sedoheptulose 7-phosphate (S7P) and (G3P) or the reverse thereof;
(iii) the conversion of S7P and G3P to ribose-5-phosphate and xylulose-5-phosphate or the reverse thereof;
(iv) the conversion of ribose-5-phosphate to ribulose-5-phosphate or the reverse thereof;
(v) the conversion of ribulose-5-phosphate to xylulose-5-phosphate or the reverse thereof;
(vi) the conversion of xylulose-5-phosphate and E4P to fructose-6-phosphate and glyceraldehyde-3-phosphate or the reverse thereof;
(vii) the conversion of glyceraldehyde-3-phosphate to dihydroxyacetone phosphate or the reverse thereof;
(viii) the conversion of dihydroxyacetone phosphate and glyceraldehyde-3-phosphate to fructose 1,6 biphosphate or the reverse thereof; and
(ix) the conversion of fructose 1,6-biphosphate to fructose-6-phosphate,
wherein the microorganism produces acetyl-phosphate, or compounds derived from acetyl-phosphate using a carbon source selected from the group consisting of a carbohydrate having the general formula (CₙH₂ₙOₙ, n=3-7), a sugar-phosphate, CO₂, CO, methanol, methane, formate, formaldehyde and any combination thereof.

39. The recombinant microorganism of claim 38, wherein the microorganism can convert a sugar phosphate to acetyl-phosphate with improved carbon yield beyond those obtained by pathways that involve pyruvate decarboxylation.

40. The recombinant microorganism of claim 38, wherein the microorganism is a prokaryote or eukaryote.

41. The recombinant microorganism of claim 38, wherein the microorganism is engineered to express a phosphoketolase.

42. The recombinant microorganism of claim 41, wherein the phosphoketolase is Fpk, Xpk or a bifunctional F/Xpk enzyme.

43. The recombinant microorganism of claim 38, wherein the microorganism is engineered to heterologously expresses one or more of the following enzymes:
(a) a phosphoketolase;
(b) a transaldolase;
(c) a transketolase;
(d) a ribose-5-phosphate isomerase;
(e) a ribulose-5-phosphate epimerase;
(f) a triose phosphate isomerase;
(g) a fructose 1,6 bisphosphate aldolase;
(h) a sedoheptulose bisphosphate aldolase;
(i) a fructose 1,6 bisphosphatase; and
(j) a sedoheptulose 1,6, bisphosphatase.

44. The recombinant microorganism of claim 41, wherein the microorganism is engineered to express a phosphoketolase derived from Bifidobaceterium adolescentis.

45. The recombinant microorganism of claim 44, wherein the phosphoketolase comprises a sequence that is at least 49% identical to SEQ ID NO:2 and has phosphoketolase activity.

46. The recombinant microorganism of claim 38, wherein the microorganism is engineered to express or over express a fructose 1,6 bisphosphatase.
